(19) United States
(12) Patent Application Publication
(10) Pub. No.: US 2025/0019368 A1
(43) Pub. Date: Jan. 16, 2025

(54) COMPOUND AND PRODUCTION METHOD THEREOF, POLYMERIZABLE COMPOSITION, RESIN COMPOSITION, POLYMER, CURED SUBSTANCE, AND LAMINATE

(71) Applicants: FUJIFILM Corporation, Tokyo (JP); FUJIFILM WAKO PURE CHEMICAL CORPORATION, Osaka (JP)

(72) Inventors: Yoshihiro JIMBO, Kanagawa (JP); Yoshihiko FUJIE, Kanagawa (JP); Jun TANABE, Kanagawa (JP); Hirotaka SATOU, Kanagawa (JP)

(73) Assignees: FUJIFILM Corporation, Tokyo (JP); FUJIFILM WAKO PURE CHEMICAL CORPORATION, Osaka (JP)

(21) Appl. No.: 18/892,564

(22) Filed: Sep. 23, 2024

Related U.S. Application Data
(63) Continuation of application No. PCT/JP2023/011604, filed on Mar. 23, 2023.

(30) Foreign Application Priority Data
Mar. 29, 2022 (JP) 2022-054280
Jul. 29, 2022 (JP) 2022-122246

(51) Int. Cl. C07D 403/08; C08J 5/18; C08K 5/3415; G02B 5/22

(52) U.S. Cl. CPC C07D 403/08; C08J 5/18; C08K 5/3415; G02B 5/223; C08J 2339/04

(57) ABSTRACT

Provided are a compound represented by Formula (X) and a production method thereof, a polymerizable composition, a resin composition, a polymer, a cured substance, and a laminate.

[in Formula (X), R¹, R³, R⁵, and R⁷ each independently represent a hydrogen atom or a substituent, R¹ and R³, R³ and R⁵, and R⁵ and R⁷ each may be bonded to each other to form a ring, and a ring A represents an aromatic ring, provided that at least one substituent in Formula (X) represents a group which includes a polymerizable group having an ethylenically unsaturated bond]

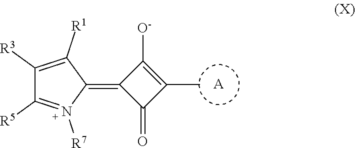

### CROSS-REFERENCE TO RELATED APPLICATIONS

This application is a continuation application of International Application No. PCT/JP2023/011604, filed on Mar. 23, 2023, the disclosure of which is incorporated herein by reference in its entirety. Further, this application claims priority from Japanese Patent Application No. 2022-054280, filed on Mar. 29, 2022, and Japanese Patent Application No. 2022-122246, filed on Jul. 29, 2022, the entire disclosure of each of which is incorporated herein by reference.

### BACKGROUND OF THE INVENTION

### 1. Field of the Invention

The present disclosure relates to a compound and a production method thereof, a polymerizable composition, a resin composition, a polymer, a cured substance, and a laminate.

### 2. Description of the Related Art

A liquid crystal display device has been widely used as a space-saving image display device with low power consumption. In a market in which a high-quality image is required, such as televisions, a demand for improvement in color reproducibility has increased in addition to a demand for resolution. The liquid crystal display device includes a non-light-emitting element which is a liquid crystal panel itself for displaying an image. Therefore, a backlight unit is disposed on a rear surface of the liquid crystal panel, and supplies light to the liquid crystal panel.

In recent years, as a light source for the backlight unit, a white light emitting diode (LED) has been used. As a light emitting method using the white LED, for example, a method of producing white light by mixing blue light radiated from a blue LED and light radiated from a yellow phosphor, or a green phosphor and a red phosphor is known. However, the white LED in the above-described method has a problem in that a color reproduction range is narrow compared to an organic light emitting diode (OLED) or the like, which has been attracting attention as a light source of a next-generation display. As a technique for solving this problem, a technique of providing a layer containing a dye on a diffusion film in the backlight unit to shield light having an unnecessary wavelength, which has been emitted from the white LED, has been proposed.

For example, JP2008-145480A, WO2008/090757A, and JP2019-12159A disclose a color adjustment filter or a color correction filter, that can selectively shield light in a wavelength range in the vicinity of 600 nm, which is transmitted through a color filter and has low color purity, by using a squarylium compound having a specific structure.

### SUMMARY OF THE INVENTION

Meanwhile, the squarylium compound in the related art tends to have deteriorated solubility in an organic solvent. In the manufacturing process of an optical filter, a photosensitive resin composition may be subjected to a step of being semi-cured by light irradiation and then being completely cured by heating. The present inventors have found that, in the manufacturing of the optical filter using a squarylium compound, in a case where a photosensitive resin composition containing the squarylium compound is irradiated with light to be semi-cured and then heated to be completely cured, optical characteristics of the optical filter to be obtained may be impaired.

The present disclosure has been made in view of the above-described circumstances.

An object to be achieved by an aspect of the present disclosure is to provide a novel compound having excellent solubility in an organic solvent as compared with a squarylium compound in the related art.

An object to be achieved by another aspect of the present disclosure is to provide a production method of the above-described compound.

An object to be achieved by still another aspect of the present disclosure is to provide a polymerizable composition and a resin composition, which contain the above-described compound; and a polymer, a cured substance, and a laminate, which are obtained using the above-described compound.

Specific methods for achieving the above-described objects include the following aspects.

<1> A compound represented by Formula (X).

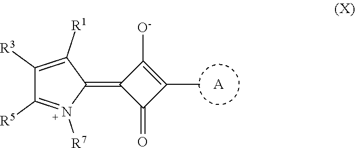

In Formula (X), R¹, R³, R⁵, and R⁷ each independently represent a hydrogen atom or a substituent, R¹ and R³, R³ and R⁵, and R⁵ and R⁷ each may be bonded to each other to form a ring, and a ring A represents an aromatic ring, provided that at least one substituent in Formula (X) represents a group which includes a polymerizable group having an ethylenically unsaturated bond.

<2> The compound according to <1>,
in which the compound represented by Formula (X) is a compound represented by Formula (1).

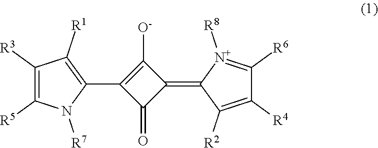

In Formula (1), R¹, R², R³, R⁴, R⁵, R⁶, R⁷, and R⁸ each independently represent a hydrogen atom or a substituent, and R¹ and R³, R² and R⁴, R³ and R⁵, R⁴ and R⁶, R⁵ and R⁷, and R⁶ and R⁸ each may be bonded to each other to form a ring, provided that at least one of R¹, R², R³, R⁴, R⁵, R⁶, R⁷, or R⁸ represents a group which includes a polymerizable group having an ethylenically unsaturated bond.

<3> The compound according to <2>,
in which, in Formula (1), R¹, R², R³, R⁴, R⁵, and R⁶ are each independently a hydrogen atom, a halogen group, an alkyl group, an aryl group, an alkoxy group, an aryloxy group, an acyl group, an alkoxycarbonyl group, an aryloxycarbonyl group, a carbamoyl group, an acylamino group, an alkoxycarbonylamino group, a carbamoylamino group, or a cyano group, R⁷ and R⁸ are each independently a hydrogen atom, an alkyl group, or an aryl group, and at least one of R¹, R², R³, R⁴, R⁵, R⁶, R⁷, or R⁸ includes the polymerizable group having an ethylenically unsaturated bond.

<4> The compound according to <2>,
in which, in Formula (1), at least one of R³ or R⁴ is a group represented by Formula (8).

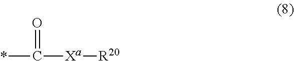

In Formula (8), X^{a} represents a single bond, an alkylene group, —O—, —S—, or —NR⁹—, where R⁹ represents a hydrogen atom or an alkyl group, R²⁰ represents a hydrogen atom or an alkyl group, and * represents a bonding position.

<5> The compound according to any one of <1> to <4>,
in which the group which includes a polymerizable group having an ethylenically unsaturated bond is a group including a group represented by Formula (2) or a group including a group represented by Formula (3).

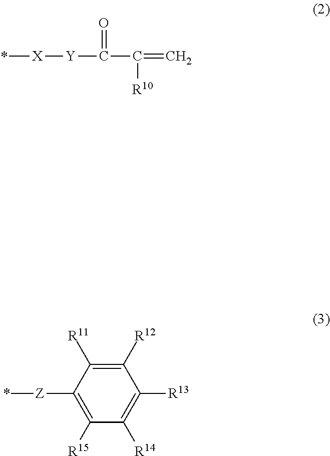

In Formula (2), X represents a single bond or an alkylene group, Y represents a single bond, —O—, or —NR¹⁶—, where R¹⁶ represents a hydrogen atom or an alkyl group, R¹⁰ represents a hydrogen atom or an alkyl group, and * represents a bonding position.

In Formula (3), R¹¹, R¹², R¹³, R¹⁴, and R¹⁵ each independently represent a hydrogen atom, a halogen group, an alkyl group, an alkoxy group, or a vinyl group, Z represents a single bond or an alkylene group, and * represents a bonding position, provided that at least one of R¹¹, R¹², R¹³, R¹⁴, R¹⁵ represents a vinyl group.

<6> The compound according to any one of <1> to <5>,
in which the compound is a squarylium coloring agent.

<7> A polymerizable composition comprising:
the compound according to any one of <1> to <6>; and a polymerizable compound.

<8> The polymerizable composition according to <7>,
in which the polymerizable compound is at least one selected from the group consisting of a (meth)acrylic acid, a (meth)acrylic acid amide compound, a (meth)acrylic acid ester compound, and a styrene compound.

<9> The polymerizable composition according to <7> or <8>, further comprising: an ultraviolet absorber.

<10> The polymerizable composition according to any one of <7> to <9>, further comprising:
a polymer compound.

<11> A resin composition comprising:
the compound according to any one of <1> to <6>; and
a resin.

<12> A polymer comprising:
a constitutional unit derived from the compound according to any one of <1> to <6>.

<13> A cured substance of the polymerizable composition according to any one of <7> to <10>.

<14> The cured substance according to <13>,
in which the cured substance is an optical filter.

<15> The cured substance according to <13> or <14>,
in which a maximal absorption wavelength is in a range of 550 nm to 610 nm.

<16> A laminate comprising:
a support; and
the cured substance according to any one of <13> to <15>.

<17> The laminate according to <16>, in which a maximal absorption wavelength is in a range of 550 nm to 610 nm.

<18> A production method of the compound according to <1>, the production method comprising:
reacting a compound represented by Formula (4) with an aromatic compound in a solvent containing an alcohol.

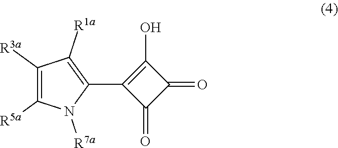

In Formula (4), R^{1a}, R^{3a}, R^{5a}, and R^{7a} each independently represent a hydrogen atom or a substituent, and R^{1a} and R^{3a}, R^{3a} and R^{5a}, and R^{5a} and R^{7a} each may be bonded to each other to form a ring, provided that at least one of R^{1a}, R^{3a}, R^{5a}, or R^{7a} represents a group which includes a polymerizable group having an ethylenically unsaturated bond.

<19> A production method of the compound according to any one of <2> to <6>, the production method comprising:
reacting a compound represented by Formula (4) with a compound represented by Formula (5) in a solvent containing an alcohol.

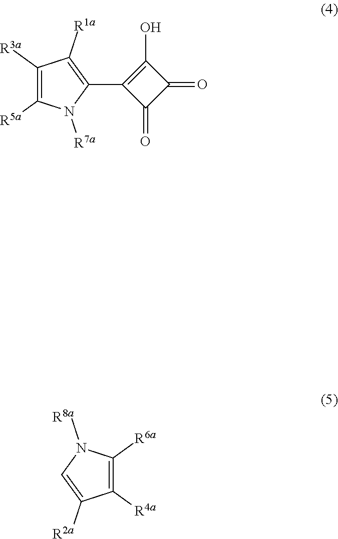

In Formula (4) and Formula (5), R^{1a}, R^{2a}, R^{3a}, R^{4a}, R^{5a}, R^{6a}, R^{7a}, and R^{8a} each independently represent a hydrogen atom or a substituent, and R^{1a} and R^{3a}, R^{2a} and R^{4a}, R^{3a} and R^{5a}, R^{4a} and R^{6a}, R^{5a} and R^{7a}, and R^{6a} and R^{8a} each may be bonded to each other to form a ring, provided that at least one of R^{1a}, R^{2a}, R^{3a}, R^{4a}, R^{5a}, R^{6a}, R^{7a}, or R^{8a} represents a group which includes a polymerizable group having an ethylenically unsaturated bond.

<20> A production method of the compound according to any one of <2> to <6>, the production method comprising:
reacting a compound represented by Formula (6) with a compound represented by Formula (7) in a solvent containing an alcohol.

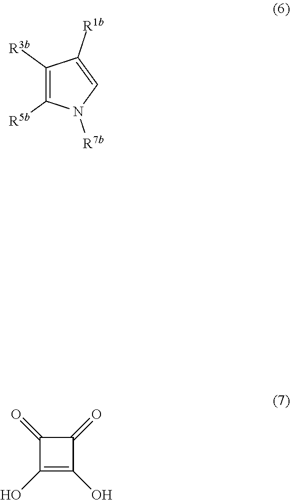

In Formula (6), R^{1b}, R^{3b}, R^{5b}, and R^{7b} each independently represent a hydrogen atom or a substituent, and R^{1b} and R^{3b}, R^{3b} and R^{5b}, and R^{5b} and R^{7b} each may be bonded to each other to form a ring, provided that at least one of R^{1b}, R^{3b}, R^{5b}, or R^{7b} represents a group which includes a polymerizable group having an ethylenically unsaturated bond.

According to the aspect of the present disclosure, there is provided a novel compound having excellent solubility in an organic solvent as compared with a squarylium compound in the related art.

According to another aspect of the present disclosure, there is provided a production method of the above-described compound.

According to still another aspect of the present disclosure, there are provided a polymerizable composition and a resin composition, which contain the above-described compound; and a polymer, a cured substance, and a laminate, which are obtained using the above-described compound.

### BRIEF DESCRIPTION OF THE DRAWINGS

FIG. 1 is a graph showing an absorption spectrum of a cured film E1.

FIG. 2 is a graph showing an absorption spectrum of a cured film E2.

FIG. 3 is a graph showing an absorption spectrum of a cured film E3.

### DESCRIPTION OF THE PREFERRED EMBODIMENTS

Hereinafter, the present disclosure will be described in detail. The description of the requirements set forth below may be based on representative embodiments of the present disclosure, but the present disclosure is not limited to such embodiments. Within the scope of the purpose of the present disclosure, modifications can be made as appropriate.

In the present disclosure, a numerical range expressed using “to” means a range including numerical values before and after “to” as a lower limit value and an upper limit value.

In a numerical range described in a stepwise manner in the present disclosure, an upper limit or a lower limit described in a certain numerical range may be replaced with an upper limit or a lower limit in another numerical range described in a stepwise manner. In addition, in a numerical range described in the present disclosure, an upper limit or a lower limit described in a certain numerical range may be replaced with a value described in Examples.

In the present disclosure, upon referring to an amount of each component in a composition, the amount means a total amount of a plurality of components present in the composition unless otherwise specified, in a case where a plurality of substances corresponding to individual components are present in the composition.

In the present disclosure, a combination of two or more preferred aspects is a more preferred aspect.

In the present disclosure, “semi-curing” means a state in which complete curing has not been carried out, and “semi-cured substance” means a cured substance in a state in which complete curing has not been carried out.

In the present disclosure, “solid content” means a component excluding a solvent, and “solvent” means water and an organic solvent.

In the present disclosure, the meaning of “step” includes not only an independent step but also a step whose intended purpose is achieved even in a case where the step is not clearly distinguished from other steps.

In the present disclosure, “(meth)acryl” is a term including both “acryl” and “methacryl”; “(meth)acrylate” is a term including both “acrylate” and “methacrylate”; “(meth)acryloyl” is a term including both “acryloyl” and “methacryloyl”; and “(meth)allyl” is a term including both “methallyl” and “allyl”.

In the present disclosure, “n-” means normal and “t-” means tertiary.

In the present disclosure, in a case where there is a molecular weight distribution, a molecular weight represents a weight-average molecular weight (Mw; the same applies hereinafter), unless otherwise noted.

The weight-average molecular weight (Mw) in the present disclosure is measured by gel permeation chromatography (GPC).

The measurement by GPC is performed using HLC (registered trademark)-8220GPC [manufactured by Tosoh Corporation] as a measuring device, three columns of TSKgel (registered trademark) Super HZ2000 [4.6 mm ID x 15 cm, manufactured by Tosoh Corporation], TSKgel (registered trademark) Super HZ4000 [4.6 mm ID x 15 cm, manufactured by Tosoh Corporation] and TSKgel (registered trademark) Super HZ-H [4.6 mm ID x 15 cm, manufactured by Tosoh Corporation] which are connected in series, and N-methylpyrrolidone (NMP) as an eluent. A differential refractive index (RI) detector as a detector is used for performing the GPC under the measurement conditions: a sample concentration of 0.3% by mass, a flow rate of 0.35 ml/min, a sample injection volume of 10 uL, and a measurement temperature of 40° C. The calibration curve is prepared using 6 samples of “F-80”, “F-20”, “F-4”, “F-2”, “A-5000”, and “A-1000”, which are “Standard Sample TSK standard, polystyrene” manufactured by Tosoh Corporation.

Regarding a term of a group (atomic group) in the present disclosure, a term with no description of “substituted” and “unsubstituted” includes both a group not having a substituent and a group having a substituent. For example, the concept of “alkyl group” includes not only an alkyl group not having a substituent (unsubstituted alkyl group) but also an alkyl group having a substituent (substituted alkyl group).

The “substituent” in the present disclosure is not particularly limited, and for example, it can be arbitrarily selected from a substituent group consisting of a halogen group, a hydroxy group, an alkyl group, a cycloalkyl group, an alkenyl group, a cycloalkenyl group, an alkynyl group, an aryl group, a heterocyclic group, an alkoxy group, an aryloxy group, a heterocyclicoxy group, a sulfo group, an acyl group, an alkoxycarbonyl group, an aryloxycarbonyl group, a carboxy group, a carbamoyl group, an acyloxy group, a carbamoyloxy group, an alkoxycarbonyloxy group, aryloxycarbonyloxy, a cyano group, a nitro group, an amino group (including an anilino group), an acylamino group, an alkoxycarbonylamino group, a carbamoylamino group, an aryloxycarbonylamino group, a sulfamoylamino group, an alkylsulfonylamino group, an arylsulfonylamino group, a mercapto group, an alkylthio group, an arylthio group, a heterocyclicthio group, a sulfamoyl group, an alkylsulfinyl group, an arylsulfinyl group, an alkylsulfonyl group, an arylsulfonyl group, an arylazo group, a heterocyclic azo group, an imide group, a phosphino group, a phosphinyl group, a phosphinyloxy group, and a phosphinylamino group.

More specific examples of the substituent in the present disclosure include: a halogen group (for example, a fluorine group, a chlorine group, a bromine group, and an iodine group); an alkyl group (a linear, branched, or cyclic alkyl group having 1 to 10, preferably 1 to 6 carbon atoms; for example, a methyl group, an ethyl group, an n-propyl group, an isopropyl group, a t-butyl group, an n-octyl group, a 2-chlorocthyl group, a 2-cyanoethyl group, and a 2-ethylhexyl group); a cycloalkyl group (preferably, a cyclopropyl group or a cyclopentyl group); an alkenyl group (a linear, branched, or cyclic alkenyl group having 2 to 10, preferably 2 to 6 carbon atoms; for example, a vinyl group, an allyl group, and a prenyl group); a cycloalkenyl group (preferably a cyclopenten-1-yl group); an alkynyl group (an alkynyl group having 2 to 10, preferably 2 to 6 carbon atoms; for example, an ethynyl group and a propargyl group); an aryl group (an aryl group having 6 to 12, preferably 6 to 8 carbon atoms; for example, a phenyl group, a p-tolyl group, a naphthyl group, a 3-chlorophenyl group, and a 2-aminophenyl group); a heterocyclic group (a monovalent group having 1 to 12, preferably 2 to 6 carbon atoms, which is obtained by removing one hydrogen atom from a 5-membered or 6-membered aromatic or non-aromatic heterocyclic compound; for example, a 1-pyrazolyl group, a 1-imidazolyl group, a 2-furyl group, a 2-thienyl group, a 4-pyrimidinyl group, and a 2-benzothiazolyl group); a cyano group; a hydroxy group; a nitro group; an alkoxy group (a linear, branched, or cyclic alkoxy group having 1 to 10, preferably 1 to 6 carbon atoms; for example, a methoxy group, an ethoxy group, an isopropoxy group, a t-butoxy group, a cyclopentyloxy group, a 2-buten-1-yloxy group, and a 2-methoxyethoxy group); an aryloxy group (an aryloxy group having 6 to 12, preferably 6 to 8 carbon atoms; for example, a phenoxy group, a 2-methylphenoxy group, a 4-t-butylphenoxy group, and a 3-nitrophenoxy group);

a heterocyclic oxy group (a heterocyclic oxy group having 1 to 12, preferably 2 to 6 carbon atoms; for example, a 1-phenyltetrazol-5-oxy-2-tetrahydropyranyloxy group); an acyloxy group (an acyloxy group having 1 to 12, preferably 1 to 8 carbon atoms; for example, a formyloxy group, an acetyloxy group, a pivaloyloxy group, a benzoyloxy group, and a p-methoxycarbonyloxy group); a carbamoyloxy group (a carbamoyloxy group having 1 to 10, preferably 1 to 6 carbon atoms; for example, an N,N-dimethylcarbamoyloxy group, an N,N-diethylcarbamoyloxy group, a morpholinocarbonyloxy group, and an N,N-octylcarbamoyloxy group); an alkoxycarbonyloxy group (an alkoxycarbonyloxy group having 2 to 10, preferably 2 to 6 carbon atoms; for example, a methoxycarbonyloxy group, an ethoxycarbonyloxy group, a t-butoxycarbonyloxy group, and an n-octyloxycarbonyloxy group); an aryloxycarbonyloxy group (an aryloxycarbonyloxy group having 7 to 12, preferably having 7 to 10 carbon atoms; for example, a phenoxycarbonyloxy group and a p-methoxyphenoxycarbonyloxy group); an amino group (an amino group, an alkylamino group having 1 to 10, preferably 1 to 6 carbon atoms, an anilino group having 6 to 12, preferably 6 to 8 carbon atoms, or a heterocyclic amino group having 1 to 12, preferably 2 to 6 carbon atoms; for example, an amino group, a methylamino group, a dimethylamino group, an anilino group, an N-methyl-anilino group, a diphenylamino group, an imidazol-2-ylamino group, and a pyrazol-3-ylamino group); an acylamino group (an alkylcarbonylamino group having 1 to 10, preferably 1 to 6 carbon atoms, an arylcarbonylamino group having 6 to 12, preferably 6 to 8 carbon atoms, or a heterocyclic carbonylamino group having 2 to 12, preferably 2 to 6 carbon atoms; for example, including a formylamino group, an acetylamino group, a pivaloylamino group, a benzoylamino group, a pyridine-4-carbonylamino group, and a thiophene-2-carbonylamino group); an aminocarbonylamino group (an aminocarbonylamino group having 1 to 12, preferably 1 to 6 carbon atoms; for example, a carbamoylamino group, an N,N-dimethylaminocarbonylamino group, an N,N-diethylaminocarbonylamino group, and a morpholin-4-ylcarbonylamino group); an alkoxycarbonylamino group (an alkoxycarbonylamino group having 2 to 10, preferably 2 to 6 carbon atoms; for example, a methoxycarbonylamino group, an ethoxycarbonylamino group, and a t-butoxycarbonylamino group);

an aryloxycarbonylamino group (an aryloxycarbonylamino group having 7 to 12, preferably 7 to 9 carbon atoms; for example, a phenoxycarbonylamino group, a p-chlorophenoxycarbonylamino group, and a 4-methoxyphenoxycarbonylamino group); a sulfamoylamino group (a sulfamoylamino group having 0 to 10, preferably 0 to 6 carbon atoms; for example, a sulfamoylamino group, an N,N-dimethylaminosulfonylamino group, and an N-(2-hydroxyethyl) sulfamoylamino group); an alkylsulfonylamino group (an alkylsulfonylamino group having 1 to 10, preferably 1 to 6 carbon atoms; for example, a methylsulfonylamino group and a butylsulfonylamino group); an arylsulfonylamino group (an arylsulfonylamino group having 6 to 12, preferably 6 to 8 carbon atoms; for example, a phenylsulfonylamino group, a 2,3,5-trichlorophenylsulfonylamino group, and p-methylphenylsulfonylamino group); a mercapto group; an alkylthio group (an alkylthio group having 1 to 10, preferably 1 to 6 carbon atoms; for example, a methylthio group, an ethylthio group, and a butylthio group); an arylthio group (an arylthio group having 6 to 12, preferably having 6 to 8 carbon atoms; for example, a phenylthio group, a p-chlorophenylthio group, and an m-methoxythio group); a heterocyclic thio group (a heterocyclic thio group having 2 to 10, preferably having 2 to 6 carbon atoms; for example, a 2-benzothiazolylthio group and a 1-phenyltetrazol-5-ylthio group); a sulfamoyl group (a sulfamoyl group having 0 to 10, preferably having 0 to 6 carbon atoms; for example, a sulfamoyl group, an N-ethylsulfamoyl group, an N,N-dimethylsulfamoyl group, an N-acetylsulfamoyl group, and an N-benzoylsulfamoyl group); an alkylsulfinyl group (an alkylsulfinyl group having 1 to 10, preferably 1 to 6 carbon atoms; for example, a methylsulfinyl group and an ethylsulfinyl group); an arylsulfinyl group (an arylsulfinyl group having 6 to 12, preferably 6 to 8 carbon atoms; for example, a phenylsulfinyl group and a p-methylphenylsulfinyl group); an alkylsulfonyl group (an alkylsulfonyl group having 1 to 10, preferably 1 to 6 carbon atoms; for example, a methylsulfonyl group and an ethylsulfonyl group); an arylsulfonyl group (an arylsulfonyl group having 6 to 12, preferably 6 to 8 carbon atoms; for example, a phenylsulfonyl group and a p-chlorophenylsulfonyl group), a sulfo group; an acyl group (a formyl group, an alkylcarbonyl group having 2 to 10 preferably 2 to 6 carbon atoms, or an arylcarbonyl group having 7 to 12, preferably 7 to 9 carbon atoms; for example, an acetyl group, a pivaloyl group, a 2-chloroacetyl group, a benzoyl group, and a 2,4-dichlorobenzoyl group);

an alkoxycarbonyl group (an alkoxycarbonyl group having 2 to 10, preferably 2 to 6 carbon atoms; for example, a methoxycarbonyl group, an ethoxycarbonyl group, a t-butoxycarbonyl group, and an isobutyloxycarbonyl group); an aryloxycarbonyl group (an aryloxycarbonyl group having 7 to 12, preferably 7 to 9, carbon atoms; for example, a phenoxycarbonyl-2-chlorophenoxycarbonyl group, a 3-nitrophenoxycarbonyl group, and a 4-t-butylphenoxycarbonyl group); a carbamoyl group (a carbamoyl group having 1 to 10, preferably 1 to 6 carbon atoms; for example, a carbamoyl group, an N-methylcarbamoyl group, an N,N-dimethylcarbamoyl group, an N-(2-hydroxyethyl) carbamoyl group, and an N-(methylsulfonyl) carbamoyl group); an arylazo group (an arylazo group having 6 to 12, preferably 6 to 8 carbon atoms; for example, a phenylazo group and a p-chlorophenylazo group); a heterocyclic azo group (a heterocyclic azo group having 1 to 10, preferably 1 to 6 carbon atoms; for example, a pyrazol-3-ylazo group, a thiazol-2-ylazo group, and a 5-methylthio-1,3,4-thiadiazol-2-ylazo group); an imido group (an imido group having 2 to 10, preferably 4 to 8 carbon atoms; for example, a succinimido group and a phthalimido group); a phosphino group (a phosphinyl group having 2 to 12, preferably 2 to 6 carbon atoms; for example, a dimethylphosphino group, a diphenylphosphino group, and a methylphenoxyphosphino group); a phosphinyl group (a phosphinyl group having 2 to 12, preferably 2 to 6 carbon atoms; for example, a phosphinyl group and a diethoxyphosphinyl group); a phosphinyloxy group (a phosphinyloxy group having 2 to 12, preferably 2 to 6 carbon atoms; for example, a diphenoxyphosphinyloxy group and a dibutoxyphosphinyloxy group); and a phosphinylamino group (a phosphinylamino group having 2 to 12, preferably 2 to 6 carbon atoms; for example, a dimethoxyphosphinylamino group a and dimethylaminophosphinylamino group).

In a case where these groups are groups which can be further substituted, these groups can further include a substituent. In a case where two or more of these groups are substituted with a substituent, the substituents may be the same or different from each other.

### [Compound]

The compound according to the present disclosure is a compound represented by Formula (X).

In a case where tautomers and/or geometric isomers exist with regard to the compound represented by Formula (X), the existing tautomers and/or geometric isomers are included in the compound represented by Formula (X).

The “tautomer” refers to, for example, a compound that exists as two or more isomers which can be readily interconverted from one to the other. Examples of the tautomer include an isomer which is generated by moving a proton bonded to one atom in a molecule to another atom, and an isomer which is generated by moving a valence localized in a specific atom in a molecule to another atom.

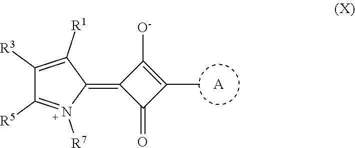

In Formula (X), R¹, R³, R⁵, and R⁷ each independently represent a hydrogen atom or a substituent, R¹ and R³, R³ and R⁵, and R⁵ and R⁷ each may be bonded to each other to form a ring, and a ring A represents an aromatic ring, provided that at least one substituent in Formula (X) represents a group which includes a polymerizable group having an ethylenically unsaturated bond.

In the present disclosure, the “polymerizable group having an ethylenically unsaturated bond” is also simply referred to as “polymerizable group”.

In the manufacturing of the optical filter using a squarylium compound, in a case where a photosensitive resin composition containing the squarylium compound is irradiated with light to be semi-cured and then heated to be completely cured, optical characteristics of the optical filter to be obtained may be impaired. The present inventors have found that the above-described problem can be solved by selecting, as the squarylium compound, a squarylium compound in which a pyrrole ring is a basic nucleus, and introducing a polymerizable group into the squarylium compound. In the related art, a squarylium compound in which a pyrrole ring substituted with a polymerizable group is a basic nucleus has not been known. The reason for this is presumed to be that it is difficult to introduce the polymerizable group into the squarylium compound in which a pyrrole ring is a basic nucleus.

As a factor of impairing the optical characteristics of the optical filter, for example, bleed-out of the squarylium compound is considerable. Since the compound according to the present disclosure has a polymerizable group, it is considered that the compound is fixed to a cured film during curing by a polymerization reaction, and thus the bleed-out is less likely to occur. In addition, the compound according to the present disclosure has excellent solubility in an organic solvent as compared with the squarylium compound in the related art. Regarding the viewpoint that the solubility in an organic solvent is excellent, it is considered that this is also one of the reasons why the bleed-out is less likely to occur in a case where the compound according to the present disclosure is used. It is presumed that the excellent solubility of the compound according to the present disclosure in an organic solvent is due to the specific structure of the compound according to the present disclosure, that is, the specific structure having a polymerizable group and having a pyrrole ring as a basic nucleus.

As described above, the present inventors have succeeded in introducing the polymerizable group into the squarylium compound in which a pyrrole ring is a basic nucleus, and have further found a new effect of the introduction of the polymerizable group, thereby completing the invention of the present disclosure.

Details of the compound represented by Formula (X) will be described.

In Formula (X), R¹, R³, R⁵, and R⁷ each independently represent a hydrogen atom or a substituent, R¹ and R³, R³ and R⁵, and R⁵ and R⁷ each may be bonded to each other to form a ring, and a ring A represents an aromatic ring, provided that at least one substituent in Formula (X) represents a group which includes a polymerizable group having an ethylenically unsaturated bond. The “substituent in Formula (X)” refers to the substituent in R¹, R³, R⁵, and R⁷ and a substituent to be substituted in the ring A.

A preferred aspect of Formula (X) is an aspect in which R¹, R³, and R⁵ are each independently a hydrogen atom, a halogen group, an alkyl group, an aryl group, an alkoxy group, an aryloxy group, an acyl group, an alkoxycarbonyl group, an aryloxycarbonyl group, a carbamoyl group, an acylamino group, an alkoxycarbonylamino group, a carbamoylamino group, or a cyano group, R⁷ is a hydrogen atom, an alkyl group, or an aryl group, the ring A is an aromatic ring, and at least one substituent in Formula (X) is the group which includes a polymerizable group having an ethylenically unsaturated bond.

As the halogen group represented by R¹, R³, and R⁵, for example, a fluoro group, a chloro group, or a bromo group is preferable.

The alkyl group represented by R¹, R³, and R⁵ may have a substituent or may not have a substituent.

The alkyl group represented by R¹, R³, and R⁵ may be a linear alkyl group, a branched alkyl group, or an alkyl group having a cyclic structure.

The alkyl group represented by R¹, R³, and R⁵ is preferably an alkyl group having 1 to 30 carbon atoms, and more preferably an alkyl group having 1 to 12 carbon atoms.

As the alkyl group represented by R¹, R³, and R⁵, for example, a methyl group, an ethyl group, an n-propyl group, an isopropyl group, an n-butyl group, a t-butyl group, an n-octyl group, a 2-cyanocthyl group, a benzyl group, a 2-ethylhexyl group, an allyl group, a prenyl group, a geranyl group, an olcyl group, a propargyl group, a cyclohexyl group, a cyclopentyl group, a 2-hydroxyethyl group, a 2-hydroxypropyl group, an acetoxymethyl group, an acryloyloxymethyl group, a methacryloyloxymethyl group, an N-(2-acryloyloxyethyl) carbamoyloxymethyl group, or an N-(2-methacryloyloxyethyl) carbamoyloxymethyl group is preferable; and a methyl group or an ethyl group is more preferable.

The aryl group represented by R¹, R³, and R⁵ may have a substituent or may not have a substituent.

The aryl group represented by R¹, R³, and R⁵ is preferably an aryl group having 6 to 30 carbon atoms, and more preferably an aryl group having 6 to 10 carbon atoms.

As the aryl group represented by R¹, R³, and R⁵, for example, a phenyl group, a p-tolyl group, or a naphthyl group is preferable.

The alkoxy group represented by R¹, R³, and R⁵ may be a linear alkoxy group, a branched alkoxy group, or an alkoxy group having a cyclic structure.

The alkoxy group represented by R¹, R³, and R⁵ is preferably an alkoxy group having 1 to 30 carbon atoms.

As the alkoxy group represented by R¹, R³, and R⁵, for example, a methoxy group or an ethoxy group is preferable.

The aryloxy group represented by R¹, R³, and R⁵ may have a substituent or may not have a substituent.

The aryloxy group represented by R¹, R³, and R⁵ is preferably an aryloxy group having 6 to 30 carbon atoms.

As the aryloxy group represented by R¹, R³, and R⁵, for example, a phenoxy group, a 2-methylphenoxy group, a 4-tert-butylphenoxy group, a 3-nitrophenoxy group, or a 2-tetradecanoylamino phenoxy group is preferable.

The acyl group represented by R¹, R³, and R⁵ is preferably an acyl group having 2 to 30 carbon atoms, and more preferably an acyl group having 2 to 15 carbon atoms.

As the acyl group represented by R¹, R³, and R⁵, for example, an acetyl group, a propionyl group, a butyryl group, an isobutyryl group, a pivaloyl group, a benzoyl group, or a 4-methoxybenzoyl group is preferable.

The alkoxycarbonyl group represented by R¹, R³, and R⁵ is preferably an alkoxycarbonyl group having 1 to 30 carbon atoms in an alkoxy moiety.

As the alkoxycarbonyl group represented by R¹, R³, and R⁵, for example, a methoxycarbonyl group, an ethoxycarbonyl group, a 2-hydroxyethoxycarbonyl group, an allyloxycarbonyl group, a 3-butenyloxycarbonyl group, a 2-acryloyloxyethoxycarbonyl group, a 2-methacryloyloxyethoxycarbonyl group, a 2-hydroxy-3-methacryloyloxypropyloxycarbonyl group, a 4-vinylbenzyloxycarbonyl group, or a 3-vinylbenzyloxycarbonyl group is preferable.

The aryloxycarbonyl group represented by R¹, R³, and R⁵ may have a substituent or may not have a substituent.

The aryloxycarbonyl group represented by R¹, R³, and R⁵ is preferably an aryloxycarbonyl group having 6 to 30 carbon atoms in an aryloxy moiety.

As the aryloxycarbonyl group represented by R¹, R³, and R⁵, for example, a phenoxycarbonyl group or a 4-methylphenoxycarbonyl group is preferable.

The carbamoyl group represented by R¹, R³, and R⁵ may have a substituent or may not have a substituent.

The carbamoyl group represented by R¹, R³, and R⁵ is preferably a carbamoyl group having 1 to 30 carbon atoms, and more preferably a carbamoyl group having 1 to 15 carbon atoms.

As the carbamoyl group represented by R¹, R³, and R⁵, for example, an unsubstituted carbamoyl group, an N-methylcarbamoyl group, an N-(2-hydroxyethyl) carbamoyl group, an N-allylcarbamoyl group, an N,N-diarylcarbamoyl group, an N-(2-acryloyloxyethyl) carbamoyl group, an N-(2-methacryloyloxyethyl) carbamoyl group, a morpholinocarbonyl group, or an N,N-bis (2-hydroxyethyl) carbamoyl group is preferable.

The acylamino group represented by R¹, R³, and R⁵ may have a substituent or may not have a substituent.

The acylamino group represented by R¹, R³, and R⁵ is preferably an acylamino group having 2 to 30 carbon atoms.

As the acylamino group represented by R¹, R³, and R⁵, for example, an acetylamino group, a propionylamino group, an acryloylamino group, or a methacryloylamino group is preferable.

The alkoxycarbonylamino group represented by R¹, R³, and R⁵ may have a substituent or may not have a substituent.

The alkoxycarbonylamino group represented by R¹, R³, and R⁵ is preferably an alkoxycarbonylamino group having 2 to 30 carbon atoms in an alkoxycarbonyl moiety.

As the alkoxycarbonylamino group represented by R¹, R³, and R⁵, for example, a methoxycarbonylamino group or an ethoxycarbonylamino group is preferable.

The carbamoylamino group represented by R¹, R³, and R⁵ may have a substituent or may not have a substituent.

The carbamoylamino group represented by R¹, R³, and R⁵ is preferably a carbamoylamino group having 1 to 30 carbon atoms.

As the carbamoylamino group represented by R¹, R³, and R⁵, for example, an unsubstituted carbamoylamino group, an N,N-dimethylcarbamoylamino group, an N-(2-acryloyloxyethyl) carbamoylamino group, or an N-(2-methacryloyloxyethyl) carbamoylamino group is preferable.

From the viewpoint of further improving the solubility in an organic solvent and in a polymerizable compound described later, for example, R¹, R³, and R⁵ are each independently more preferably a hydrogen atom, an alkyl group, or an alkoxycarbonyl group, and particularly preferably a methyl group or an ethyl group.

R³ is preferably a group represented by Formula (8).

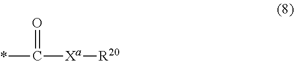

In Formula (8), X^{a} represents a single bond, an alkylene group, —O—, —S—, or —NR⁹—, where R⁹ represents a hydrogen atom or an alkyl group, R²⁰ represents a hydrogen atom or an alkyl group, and
* represents a bonding position.

The alkylene group represented by X^{a} may be a linear alkylene group, a branched alkylene group, or an alkylene group having a cyclic structure.

The alkylene group represented by X^{a} is preferably an alkylene group having 1 to 20 carbon atoms, and more preferably an alkylene group having 1 to 8 carbon atoms.

For example, the alkylene group represented by X^{a} may be unsubstituted, may have a substituent such as a methyl group, an ethyl group, and a hydroxy group, or may be interrupted by an oxygen atom and/or a sulfur atom.

As the alkylene group represented by X^{a}, for example, a methylene group, an ethylene group, a methylethylene group, an n-propylene group, a 2-hydroxypropylene group, a tetramethylene group, a hexamethylene group, an octamethylene group, —(OCH₂CH₂)ₙ—, or (OCH₂CHCH₃)ₙ— is preferable. n represents an integer of 1 to 4.

The alkyl group represented by R⁹ may have a substituent or may not have a substituent.

The alkyl group represented by R⁹ may be a linear alkyl group, a branched alkyl group, or an alkyl group having a cyclic structure.

The alkyl group represented by R⁹ is preferably an alkyl group having 1 to 8 carbon atoms.

As the alkyl group represented by R⁹, for example, a methyl group, an ethyl group, an n-propyl group, an n-butyl group, a hexyl group, or an n-octyl group is preferable.

The alkyl group represented by R²⁰ may have a substituent or may not have a substituent.

The alkyl group represented by R²⁰ may be a linear alkyl group, a branched alkyl group, or an alkyl group having a cyclic structure.

The alkyl group represented by R²⁰ is preferably an alkyl group having 1 to 8 carbon atoms.

As the alkyl group represented by R²⁰, for example, a methyl group or a methoxymethyl group is preferable.

From the viewpoint of further improving polymerization reactivity, for example, R²⁰ is preferably a hydrogen atom or a methyl group.

The alkyl group represented by R⁷ may have a substituent or may not have a substituent.

The alkyl group represented by R⁷ may be a linear alkyl group, a branched alkyl group, or an alkyl group having a cyclic structure.

The alkyl group represented by R⁷ is preferably an alkyl group having 1 to 22 carbon atoms.

As the alkyl group represented by R⁷, for example, a methyl group, an ethyl group, an n-propyl group, a 2-methacryloyloxyethyl group, a 3-methacryloyloxypropyl group, a benzyl group, a 4-fluorobenzyl group, a 4-vinylbenzyl group, or a 3-vinylbenzyl group is preferable. The aryl group represented by R⁷ may have a substituent or may not have a substituent.

The aryl group represented by R⁷ is preferably an aryl group having 6 to 20 carbon atoms, and more preferably an aryl group having 6 to 10 carbon atoms.

As the aryl group represented by R⁷, for example, a phenyl group, a 4-chlorophenyl group, or a 4-methoxyphenyl group is preferable.

From the viewpoint of further improving the solubility in an organic solvent and in a polymerizable compound described later, for example, R⁷ is preferably a hydrogen atom or an aryl group.

R¹ and R³, R³ and R⁵, and R⁵ and R⁷ each may be bonded to each other to form a ring.

The ring to be formed may be a saturated ring or an unsaturated ring.

The ring to be formed is preferably a 5-membered ring or a 6-membered ring, and more preferably a 5-membered or 6-membered unsaturated ring. The 5-membered or 6-membered ring may be further fused.

As the ring to be formed, for example, a cyclopentene ring, a cyclohexene ring, or a benzene ring is preferable.

The aromatic ring represented by the ring A may be an aromatic hydrocarbon ring or an aromatic heterocyclic ring.

The aromatic ring represented by the ring A may be a monocyclic ring or a polycyclic ring.

The aromatic ring represented by the ring A may be formed by condensing two or more aromatic hydrocarbon rings, may be formed by condensing two or more aromatic heterocyclic rings, or may be formed by condensing one or more aromatic hydrocarbon rings and one or more aromatic heterocyclic rings.

The aromatic ring represented by the ring A may have a substituent or may not have a substituent.

In a case where the aromatic ring represented by the ring A is an aromatic hydrocarbon ring, the number of carbon atoms in the aromatic hydrocarbon ring is preferably 6 to 20 and more preferably 6 to 10.

In a case where the aromatic ring represented by the ring A is an aromatic hydrocarbon ring, the aromatic hydrocarbon ring is, for example, preferably a benzene ring.

In a case where the aromatic ring represented by the ring A is an aromatic heterocyclic ring, the aromatic heterocyclic ring is preferably a 5-membered ring or a 6-membered ring.

In a case where the aromatic ring represented by the ring A is an aromatic heterocyclic ring, the aromatic heterocyclic ring preferably includes at least one heteroatom selected from the group consisting of an oxygen atom, a sulfur atom, and a nitrogen atom in the ring; more preferably includes at least one heteroatom selected from a sulfur atom or a nitrogen atom in the ring; and still more preferably includes a nitrogen atom.

In a case where the aromatic ring represented by the ring A is an aromatic heterocyclic ring, the aromatic heterocyclic ring is, for example, preferably a thiophene ring, an imidazole ring, or a pyridine ring.

At least one substituent in Formula (X) represents a group which includes a polymerizable group having an ethylenically unsaturated bond.

The polymerizable group having an ethylenically unsaturated bond is not particularly limited, and examples thereof include a vinyl group, a (meth)allyl group, a (meth)acryloyl group, a (meth)acryloyloxy group, a (meth)acryloylamino group, and a vinylphenyl group.

The group which includes a polymerizable group having an ethylenically unsaturated bond is not particularly limited, but for example, preferably a group including a group represented by Formula (2) or a group including a group represented by Formula (3).

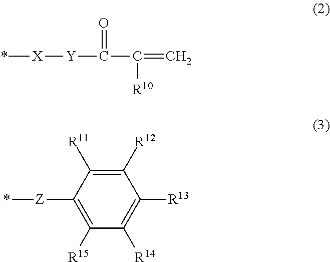

In Formula (2), X represents a single bond or an alkylene group, Y represents a single bond, —O—, or —NR¹⁶—, where R¹⁶ represents a hydrogen atom or an alkyl group, R¹⁰ represents a hydrogen atom or an alkyl group, and * represents a bonding position.

In Formula (3), R¹¹, R¹², R¹³, R¹⁴, and R¹⁵ each independently represent a hydrogen atom, a halogen group, an alkyl group, an alkoxy group, or a vinyl group, Z represents a single bond or an alkylene group, and * represents a bonding position, provided that at least one of R¹¹, R¹², R¹³, R¹⁴, R¹⁵ represents a vinyl group.

In Formula (2), X represents a single bond or an alkylene group.

The alkylene group represented by X may be a linear alkylene group, a branched alkylene group, or an alkylene group having a cyclic structure.

The alkylene group represented by X is preferably an alkylene group having 1 to 20 carbon atoms, and more preferably an alkylene group having 1 to 8 carbon atoms.

For example, the alkylene group represented by X may be unsubstituted, may have a substituent such as a methyl group, an ethyl group, and a hydroxy group, or may be interrupted by an oxygen atom and/or a sulfur atom.

As the alkylene group represented by X, for example, a methylene group, an ethylene group, a methylethylene group, an n-propylene group, a 2-hydroxypropylene group, a tetramethylene group, a hexamethylene group, an octamethylene group, —(OCH₂CH₂)ₙ—, or (OCH₂CHCH₃)ₙ— is preferable. n represents an integer of 1 to 4.

In Formula (2), Y represents a single bond, —O—, or —NR¹⁶—, where R¹⁶ represents a hydrogen atom or an alkyl group.

The alkyl group represented by R¹⁶ may have a substituent or may not have a substituent.

The alkyl group represented by R¹⁶ may be a linear alkyl group, a branched alkyl group, or an alkyl group having a cyclic structure.

The alkyl group represented by R¹⁶ is preferably an alkyl group having 1 to 8 carbon atoms.

As the alkyl group represented by R¹⁶, for example, a methyl group, an ethyl group, an n-propyl group, an n-butyl group, a hexyl group, or an n-octyl group is preferable.

In Formula (2), R¹⁰ represents a hydrogen atom or an alkyl group.

The alkyl group represented by R¹⁰ may have a substituent or may not have a substituent.

The alkyl group represented by R¹⁰ may be a linear alkyl group, a branched alkyl group, or an alkyl group having a cyclic structure.

The alkyl group represented by R¹⁰ is preferably an alkyl group having 1 to 8 carbon atoms.

As the alkyl group represented by R¹⁰, for example, a methyl group or a methoxymethyl group is preferable.

From the viewpoint of further improving polymerization reactivity, for example, R¹⁰ is preferably a hydrogen atom or a methyl group.

In Formula (3), R¹¹, R¹², R¹³, R¹⁴, and R¹⁵ each independently represent a hydrogen atom, a halogen group, an alkyl group, or an alkoxy group. Here, at least one of R¹¹, R¹², R¹³, R¹⁴, R¹⁵ represents a vinyl group.

As the halogen group represented by R¹¹, R¹², R¹³, R¹⁴, and R¹⁵, for example, a fluoro group, a chloro group, a bromo group, or an iodo group is preferable; and a fluoro group, a chloro group, or a bromo group is more preferable.

The alkyl group represented by R¹¹, R¹², R¹³, R¹⁴, and R¹⁵ may have a substituent or may not have a substituent.

The alkyl group represented by R¹¹, R¹², R¹³, R¹⁴, and R¹⁵ may be a linear alkyl group, a branched alkyl group, or an alkyl group having a cyclic structure.

The alkyl group represented by R¹¹, R¹², R¹³, R¹⁴, and R¹⁵ is preferably an alkyl group having 1 to 8 carbon atoms.

As the alkyl group represented by R¹¹, R¹², R¹³, R¹⁴, and R¹⁵, for example, a methyl group or an ethyl group is preferable.

The alkoxy group represented by R¹¹, R¹², R¹³, R¹⁴, and R¹⁵ may be a linear alkoxy group, a branched alkoxy group, or an alkoxy group having a cyclic structure.

The alkoxy group represented by R¹¹, R¹², R¹³, R¹⁴, and R¹⁵ is preferably an alkoxy group having 1 to 30 carbon atoms.

As the alkoxy group represented by R¹¹, R¹², R¹³, R¹⁴, and R¹⁵, for example, a methoxy group, an ethoxy group, or a methylenedioxy group is preferable.

At least one of R¹¹, R¹², R¹³, R¹⁴, R¹⁵ represents a vinyl group.

It is preferable that any one of R¹¹, R¹², R¹³, R¹⁴, or R¹⁵ is a vinyl group and the other four are hydrogen atoms.

In Formula (3), Z represents a single bond or an alkylene group.

The alkylene group represented by Z may be a linear alkylene group, a branched alkylene group, or an alkylene group having a cyclic structure.

The alkylene group represented by Z is preferably an alkylene group having 1 to 20 carbon atoms, and more preferably an alkylene group having 1 to 8 carbon atoms.

For example, the alkylene group represented by Z may be unsubstituted, may have a substituent such as a methyl group, an ethyl group, and a hydroxy group, or may be interrupted by an oxygen atom and/or a sulfur atom.

As the alkylene group represented by Z, for example, a methylene group, an ethylene group, a methylethylene group, an n-propylene group, a 2-hydroxypropylene group, a tetramethylene group, a hexamethylene group, an octamethylene group, —(OCH₂CH₂)ₙ—, or (OCH₂CHCH₃)ₙ— is preferable. n represents an integer of 1 to 4.

The compound represented by Formula (X) is preferably a compound represented by Formula (1).

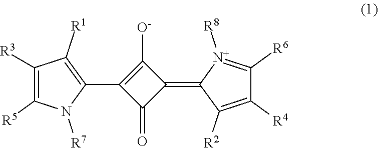

In Formula (1), R¹, R², R³, R⁴, R⁵, R⁶, R⁷, and R⁸ each independently represent a hydrogen atom or a substituent, and R¹ and R³, R² and R⁴, R³ and R⁵, R⁴ and R⁶, R⁵ and R⁷, and R⁶ and R⁸ each may be bonded to each other to form a ring, provided that at least one of R¹, R², R³, R⁴, R⁵, R⁶, R⁷, or R⁸ represents a group which includes a polymerizable group having an ethylenically unsaturated bond.

Details of the compound represented by Formula (1) will be described.

A preferred aspect of Formula (1) is an aspect in which R¹, R², R³, R⁴, R⁵, and R⁶ are each independently a hydrogen atom, a halogen group, an alkyl group, an aryl group, an alkoxy group, an aryloxy group, an acyl group, an alkoxycarbonyl group, an aryloxycarbonyl group, a carbamoyl group, an acylamino group, an alkoxycarbonylamino group, a carbamoylamino group, or a cyano group, R⁷ and R⁸ are each independently a hydrogen atom, an alkyl group, or an aryl group, and at least one of R¹, R², R³, R⁴, R⁵, R⁶, R⁷, or R⁸ includes the polymerizable group having an ethylenically unsaturated bond.

A more preferred aspect of Formula (1) is an aspect in which R¹, R², R³, R⁴, R⁵, and R⁶ are each independently a halogen group, an alkyl group, an aryl group, an alkoxy group, an aryloxy group, an acyl group, an alkoxycarbonyl group, an aryloxycarbonyl group, an acylamino group, an alkoxycarbonylamino group, a carbamoylamino group, or a cyano group, R⁷ and R⁸ are each independently a hydrogen atom or an alkyl group, and at least one of R¹, R², R³, R⁴, R⁵, R⁶, R⁷, or R⁸ includes the polymerizable group having an ethylenically unsaturated bond.

R¹, R³, R⁵, and R⁷ in Formula (1) have the same meanings as R¹, R³, R⁵, and R⁷ in Formula (X), respectively, and preferred aspects thereof are also the same, so that the description thereof will be omitted here.

As the halogen group represented by R², R⁴, and R⁶, for example, a fluoro group, a chloro group, or a bromo group is preferable.

The alkyl group represented by R², R⁴, and R⁶ may have a substituent or may not have a substituent.

The alkyl group represented by R², R⁴, and R⁶ may be a linear alkyl group, a branched alkyl group, or an alkyl group having a cyclic structure.

The alkyl group represented by R², R⁴, and R⁶ is preferably an alkyl group having 1 to 30 carbon atoms, and more preferably an alkyl group having 1 to 12 carbon atoms.

As the alkyl group represented by R², R⁴, and R⁶, for example, a methyl group, an ethyl group, an n-propyl group, an isopropyl group, an n-butyl group, a t-butyl group, an n-octyl group, a 2-cyanoethyl group, a benzyl group, a 2-ethylhexyl group, an allyl group, a prenyl group, a geranyl group, an oleyl group, a propargyl group, a cyclohexyl group, a cyclopentyl group, a 2-hydroxyethyl group, a 2-hydroxypropyl group, an acetoxymethyl group, an acryloyloxymethyl group, a methacryloyloxymethyl group, an N-(2-acryloyloxyethyl) carbamoyloxymethyl group, or an N-(2-methacryloyloxyethyl) carbamoyloxymethyl group is preferable; and a methyl group or an ethyl group is more preferable.

The aryl group represented by R², R⁴, and R⁶ may have a substituent or may not have a substituent.

The aryl group represented by R², R⁴, and R⁶ is preferably an aryl group having 6 to 30 carbon atoms, and more preferably an aryl group having 6 to 10 carbon atoms.

As the aryl group represented by R², R⁴, and R⁶, for example, a phenyl group, a p-tolyl group, or a naphthyl group is preferable.

The alkoxy group represented by R², R⁴, and R⁶ may be a linear alkoxy group, a branched alkoxy group, or an alkoxy group having a cyclic structure.

The alkoxy group represented by R², R⁴, and R⁶ is preferably an alkoxy group having 1 to 30 carbon atoms.

As the alkoxy group represented by R², R⁴, and R⁶, for example, a methoxy group or an ethoxy group is preferable.

The aryloxy group represented by R², R⁴, and R⁶ may have a substituent or may not have a substituent.

The aryloxy group represented by R², R⁴, and R⁶ is preferably an aryloxy group having 6 to 30 carbon atoms.

As the aryloxy group represented by R², R⁴, and R⁶, for example, a phenoxy group, a 2-methylphenoxy group, a 4-tert-butylphenoxy group, a 3-nitrophenoxy group, or a 2-tetradecanoylamino phenoxy group is preferable.

The acyl group represented by R², R⁴, and R⁶ is preferably an acyl group having 2 to 30 carbon atoms, and more preferably an acyl group having 2 to 15 carbon atoms.

As the acyl group represented by R², R⁴, and R⁶, for example, an acetyl group, a propionyl group, a butyryl group, an isobutyryl group, a pivaloyl group, a benzoyl group, or a 4-methoxybenzoyl group is preferable.

The alkoxycarbonyl group represented by R², R⁴, and R⁶ is preferably an alkoxycarbonyl group having 1 to 30 carbon atoms in an alkoxy moiety.

As the alkoxycarbonyl group represented by R², R⁴, and R⁶, for example, a methoxycarbonyl group, an ethoxycarbonyl group, a 2-hydroxyethoxycarbonyl group, an allyloxycarbonyl group, a 3-butenyloxycarbonyl group, a 2-acryloyloxyethoxycarbonyl group, a 2-methacryloyloxyethoxycarbonyl group, a 2-hydroxy-3-methacryloyloxypropyloxycarbonyl group, a 4-vinylbenzyloxycarbonyl group, or a 3-vinylbenzyloxycarbonyl group is preferable.

The aryloxycarbonyl group represented by R², R⁴, and R⁶ may have a substituent or may not have a substituent.

The aryloxycarbonyl group represented by R², R⁴, and R⁶ is preferably an aryloxycarbonyl group having 6 to 30 carbon atoms in an aryloxy moiety.

As the aryloxycarbonyl group represented by R², R⁴, and R⁶, for example, a phenoxycarbonyl group or a 4-methylphenoxycarbonyl group is preferable.

The carbamoyl group represented by R², R⁴, and R⁶ may have a substituent or may not have a substituent.

The carbamoyl group represented by R², R⁴, and R⁶ is preferably a carbamoyl group having 1 to 30 carbon atoms, and more preferably a carbamoyl group having 1 to 15 carbon atoms.

As the carbamoyl group represented by R², R⁴, and R⁶, for example, an unsubstituted carbamoyl group, an N-methylcarbamoyl group, an N-(2-hydroxyethyl) carbamoyl group, an N-allylcarbamoyl group, an N,N-diarylcarbamoyl group, an N-(2-acryloyloxyethyl) carbamoyl group, an N-(2-methacryloyloxyethyl) carbamoyl group, a morpholinocarbonyl group, or an N,N-bis (2-hydroxyethyl) carbamoyl group is preferable.

The acylamino group represented by R², R⁴, and R⁶ may have a substituent or may not have a substituent.

The acylamino group represented by R², R⁴, and R⁶ is preferably an acylamino group having 2 to 30 carbon atoms.

As the acylamino group represented by R², R⁴, and R⁶, for example, an acetylamino group, a propionylamino group, an acryloylamino group, or a methacryloylamino group is preferable.

The alkoxycarbonylamino group represented by R², R⁴, and R⁶ may have a substituent or may not have a substituent.

The alkoxycarbonylamino group represented by R², R⁴, and R⁶ is preferably an alkoxycarbonylamino group having 2 to 30 carbon atoms in an alkoxycarbonyl moiety.

As the alkoxycarbonylamino group represented by R², R⁴, and R⁶, for example, a methoxycarbonylamino group or an ethoxycarbonylamino group is preferable.

The carbamoylamino group represented by R², R⁴, and R⁶ may have a substituent or may not have a substituent.

The carbamoylamino group represented by R², R⁴, and R⁶ is preferably a carbamoylamino group having 1 to 30 carbon atoms.

As the carbamoylamino group represented by R², R⁴, and R⁶, for example, an unsubstituted carbamoylamino group, an N,N-dimethylcarbamoylamino group, an N-(2-acryloyloxyethyl) carbamoylamino group, or an N-(2-methacryloyloxyethyl) carbamoylamino group is preferable.

From the viewpoint of further improving the solubility in an organic solvent and in a polymerizable compound described later, for example, R², R⁴, and R⁶ are each independently more preferably a hydrogen atom, an alkyl group, or an alkoxycarbonyl group, and particularly preferably a methyl group or an ethyl group.

The alkyl group represented by R⁸ may have a substituent or may not have a substituent.

The alkyl group represented by R⁸ may be a linear alkyl group, a branched alkyl group, or an alkyl group having a cyclic structure.

The alkyl group represented by R⁸ is preferably an alkyl group having 1 to 22 carbon atoms.

As the alkyl group represented by R⁸, for example, a methyl group, an ethyl group, an n-propyl group, a 2-methacryloyloxyethyl group, a 3-methacryloyloxypropyl group, a benzyl group, a 4-fluorobenzyl group, a 4-vinylbenzyl group, or a 3-vinylbenzyl group is preferable.

The aryl group represented by R⁸ may have a substituent or may not have a substituent.

The aryl group represented by R⁸ is preferably an aryl group having 6 to 20 carbon atoms, and more preferably an aryl group having 6 to 10 carbon atoms.

As the aryl group represented by R⁸, for example, a phenyl group, a 4-chlorophenyl group, or a 4-methoxyphenyl group is preferable.

From the viewpoint of raw material availability and productivity, it is preferable that at least one of R³ or R⁴ is the group represented by Formula (8) described above.

The group represented by Formula (8) is as described above, and thus the description thereof will be omitted here.

R¹ and R³, R² and R⁴, R³ and R⁵, R⁴ and R⁶, R⁵ and R⁷, and R⁶ and R⁸ each may be bonded to each other to form a ring.

The ring to be formed may be a saturated ring or an unsaturated ring.

The ring to be formed is preferably a 5-membered ring or a 6-membered ring, and more preferably a 5-membered or 6-membered unsaturated ring. The 5-membered or 6-membered ring may be further fused.

As the ring to be formed, for example, a cyclopentene ring, a cyclohexene ring, or a benzene ring is preferable.

At least one of R¹, R², R³, R⁴, R⁵, R⁶, R⁷, or R⁸ represents a group which includes a polymerizable group having an ethylenically unsaturated bond.

The polymerizable group having an ethylenically unsaturated bond is not particularly limited, and examples thereof include a vinyl group, a (meth)allyl group, a (meth)acryloyl group, a (meth)acryloyloxy group, a (meth)acryloylamino group, and a vinylphenyl group.

The group which includes a polymerizable group having an ethylenically unsaturated bond is not particularly limited, but for example, preferably a group including the group represented by Formula (2) or a group including the group represented by Formula (3).

The group including the group represented by Formula (2) or the group including the group represented by Formula (3) is as described above, and thus the description thereof will be omitted here.

An aspect of the compound represented by Formula (1) is that R¹ is a hydrogen atom or an unsubstituted alkyl group, R² is a hydrogen atom or an unsubstituted alkyl group, R³ is the group which includes a polymerizable group having an ethylenically unsaturated bond, R⁴ is the group which includes a polymerizable group having an ethylenically unsaturated bond, R⁵ is a hydrogen atom, an unsubstituted alkyl group, or an unsubstituted aryl group, R⁶ is a hydrogen atom, an unsubstituted alkyl group, or an aryl group which may be substituted, R⁷ is an unsubstituted alkyl group, and R⁸ is an unsubstituted alkyl group.

In addition, an aspect of the compound represented by Formula (1) is that R¹ is a hydrogen atom or an unsubstituted alkyl group, R² is a hydrogen atom or an unsubstituted alkyl group, R³ is an unsubstituted alkyl group, R⁴ is an unsubstituted alkyl group, R⁵ is the group which includes a polymerizable group having an ethylenically unsaturated bond, R⁶ is the group which includes a polymerizable group having an ethylenically unsaturated bond, R⁷ is an unsubstituted alkyl group, and R⁸ is an unsubstituted alkyl group.

In addition, an aspect of the compound represented by Formula (1) is that RI is a hydrogen atom or an unsubstituted alkyl group, R² is a hydrogen atom or an unsubstituted alkyl group, R³ is an unsubstituted alkyl group, R⁴ is an unsubstituted alkyl group, R⁵ is a hydrogen atom, an unsubstituted alkyl group, or an unsubstituted aryl group, R⁶ is a hydrogen atom, an unsubstituted alkyl group, or an aryl group which may be substituted, R⁷ is the group which includes a polymerizable group having an ethylenically unsaturated bond, and R⁸ is the group which includes a polymerizable group having an ethylenically unsaturated bond.

A preferred aspect of the compound represented by Formula (1) are an aspect in which R¹ is an unsubstituted alkyl group, R² is an unsubstituted alkyl group, R³ is an unsubstituted alkyl group or the group including the group represented by Formula (2), R⁴ is an unsubstituted alkyl group or the group including the group represented by Formula (2), R⁵ is an unsubstituted alkyl group, R⁶ is an unsubstituted alkyl group, R⁷ is a hydrogen atom or the group including the group represented by Formula (3), and R⁸ is a hydrogen atom or the group including the group represented by Formula (3).

In addition, a more preferred aspect of the compound represented by Formula (1) is an aspect in which R¹ is an unsubstituted alkyl group having 1 to 4 carbon atoms, R² is an unsubstituted alkyl group having 1 to 4 carbon atoms, R³ is a methyl group, an ethyl group, or the group including the group represented by Formula (2), R⁴ is a methyl group, an ethyl group, or the group including the group represented by Formula (2), R⁵ is a hydrogen atom or a methyl group, R⁶ is a hydrogen atom or a methyl group, R⁷ is the group including the group represented by Formula (3), and R⁸ is the group including the group represented by Formula (3).

Specific examples of the compound represented by Formula (X) (so-called, exemplary compounds) are shown below, but the present disclosure is not limited to these examples. “Me” represents a methyl group, “Et” represents an ethyl group, and “tBu” represents a t-butyl group (the same applies hereinafter).

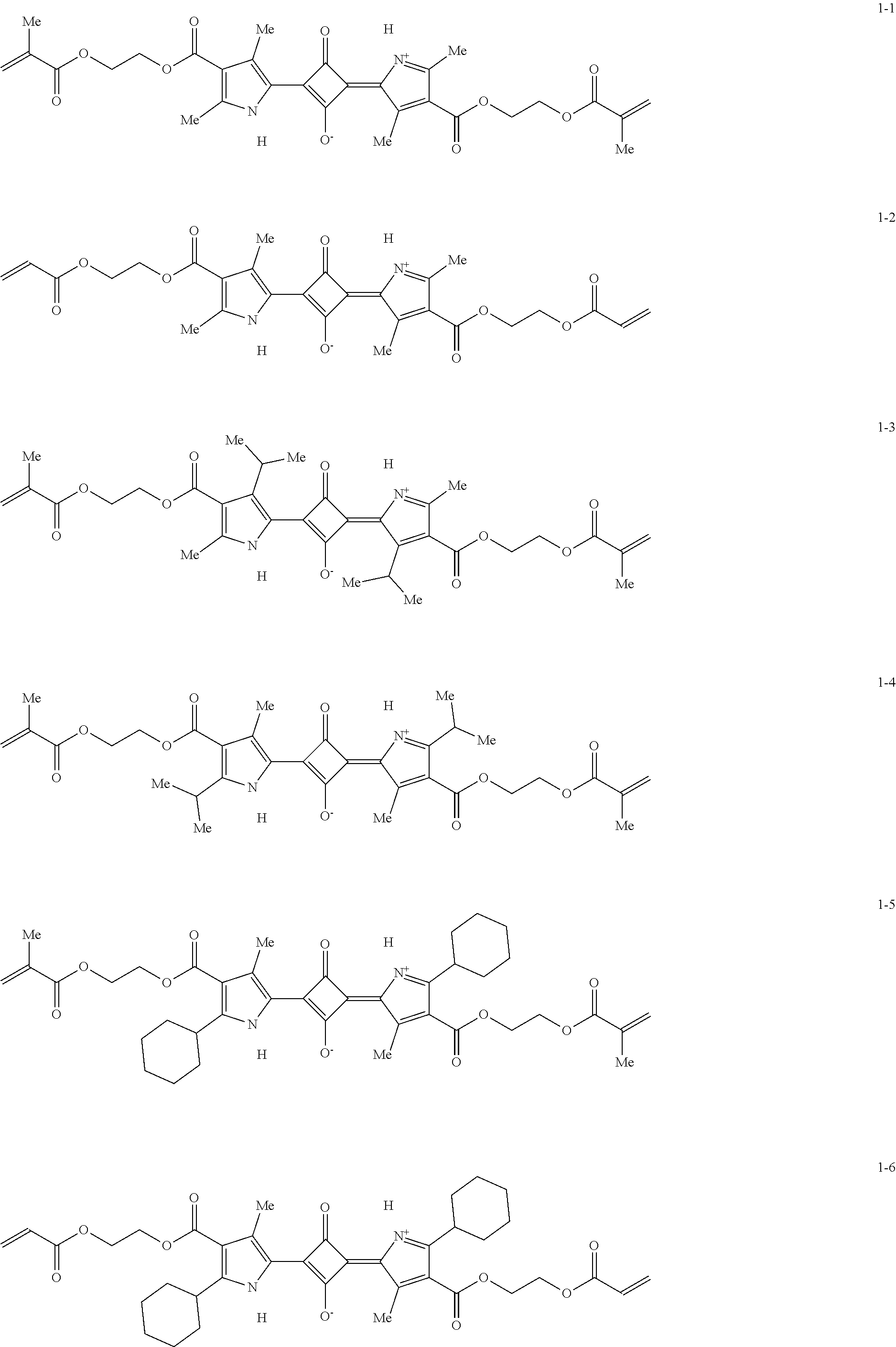

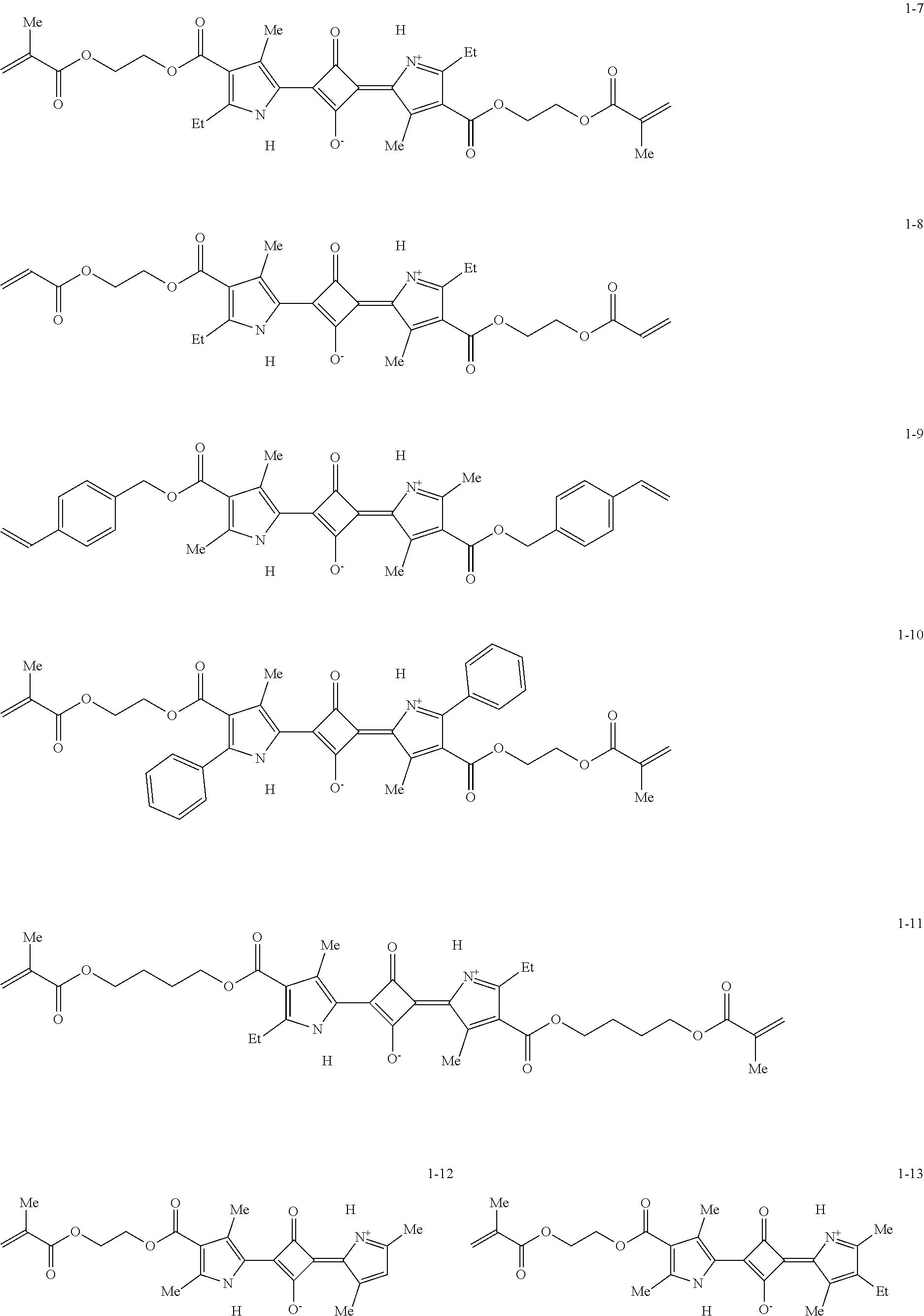

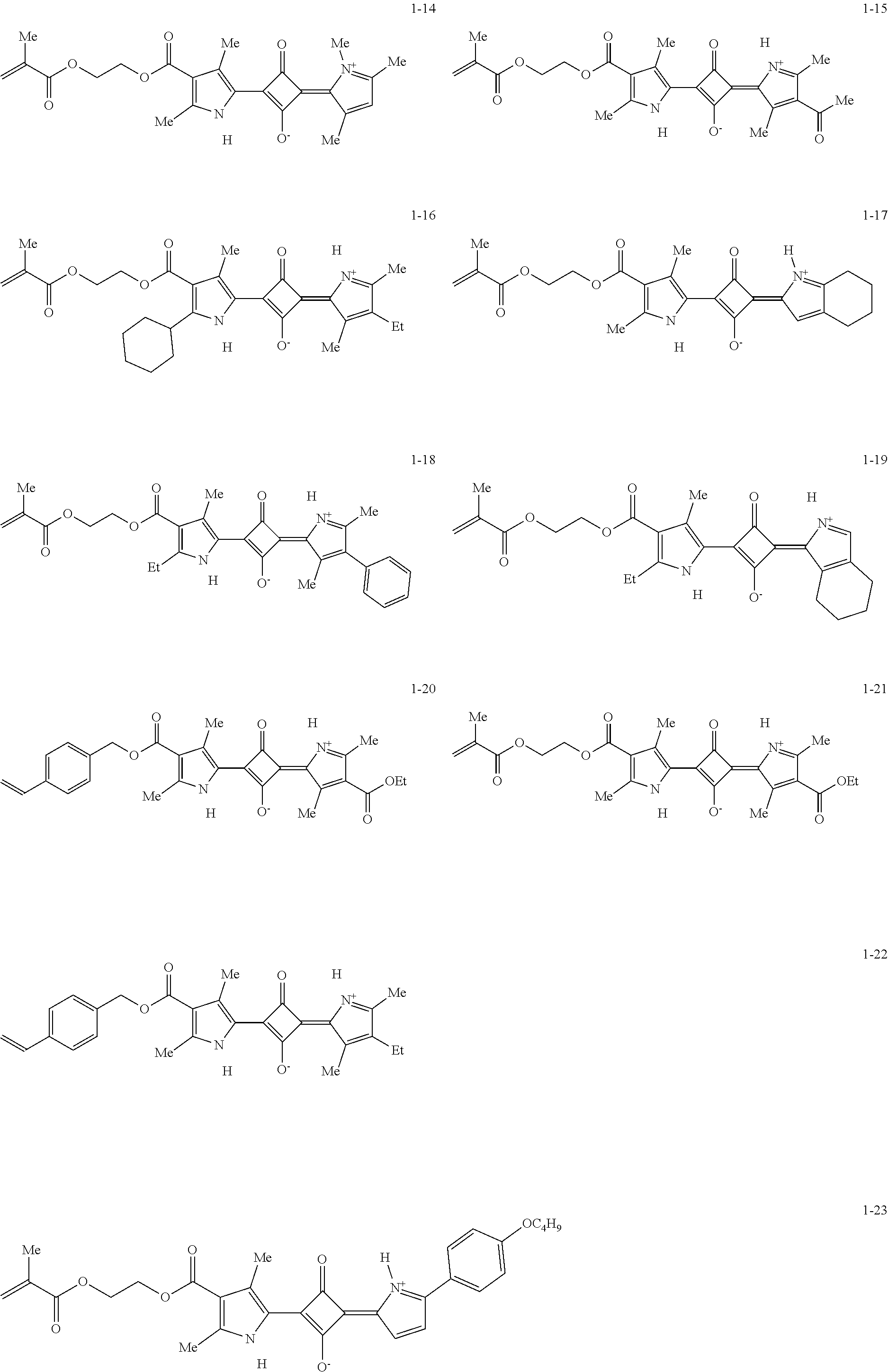

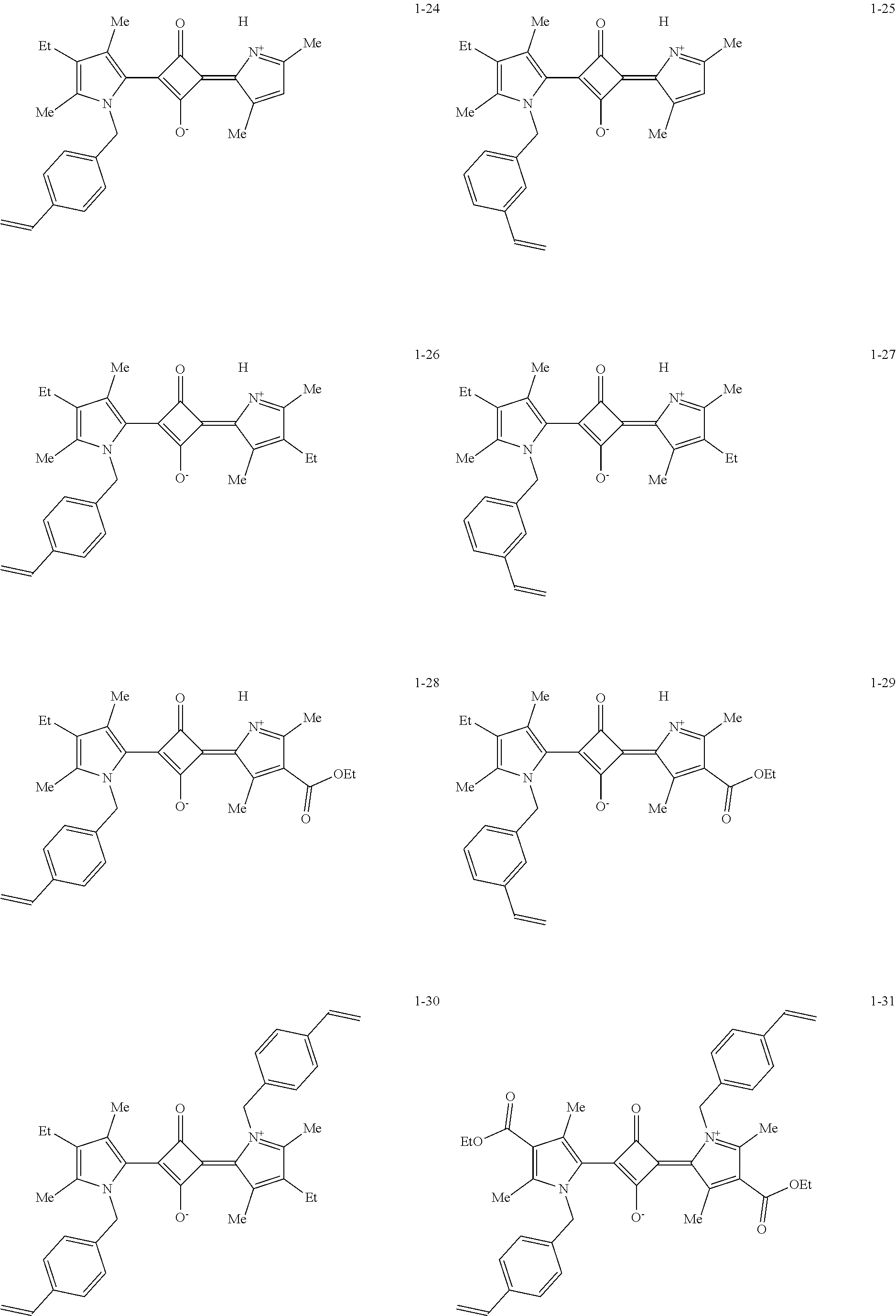

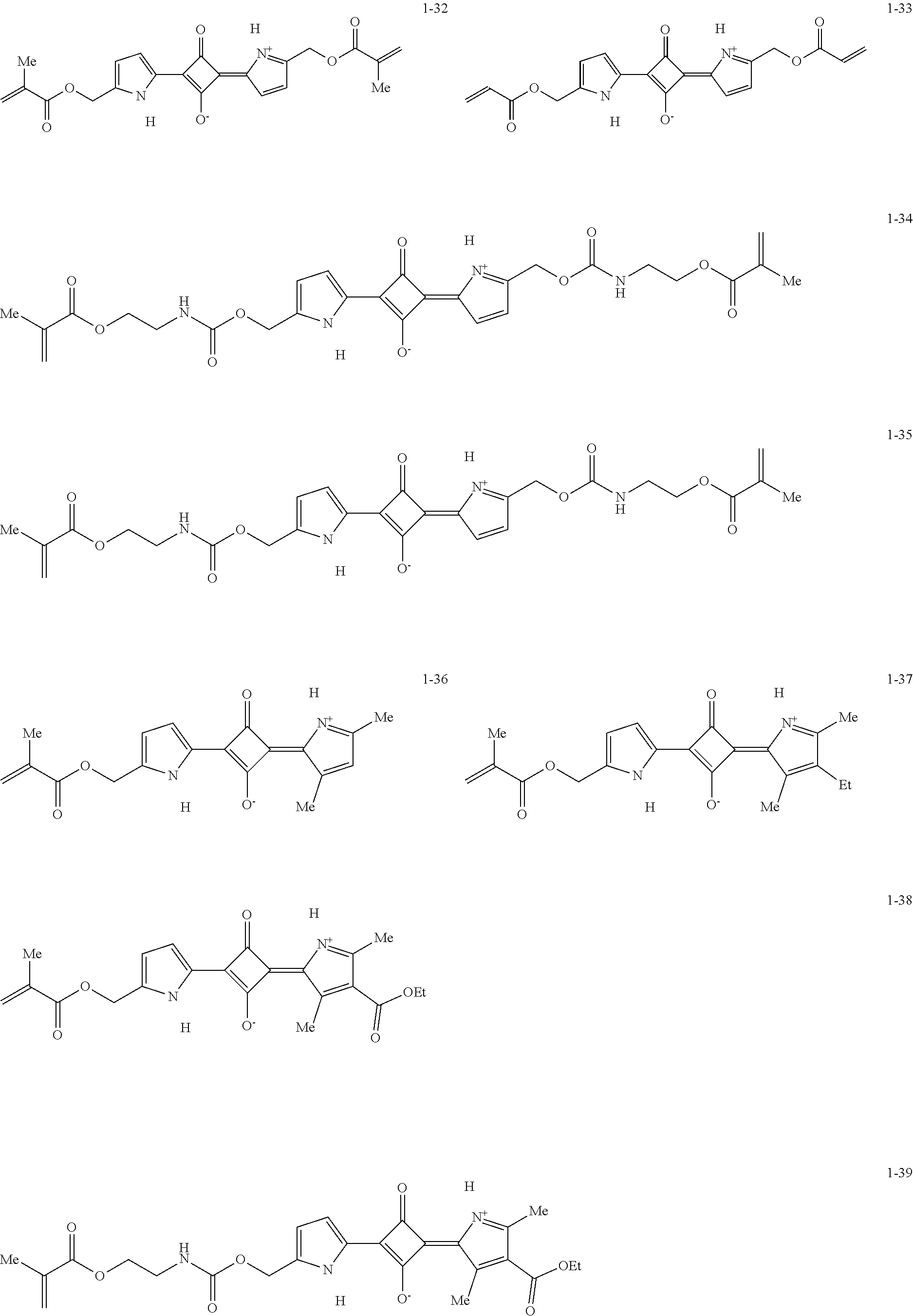

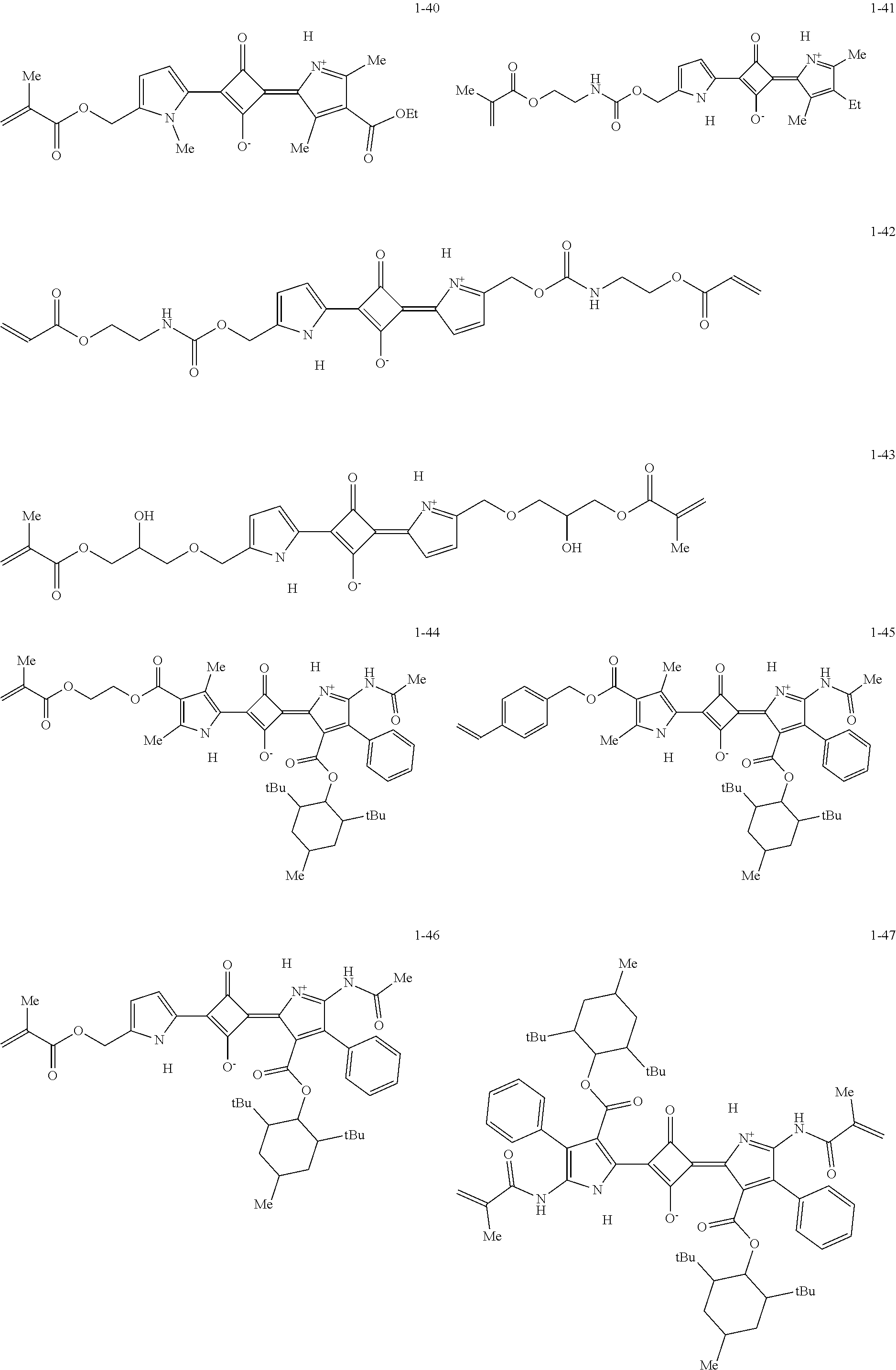

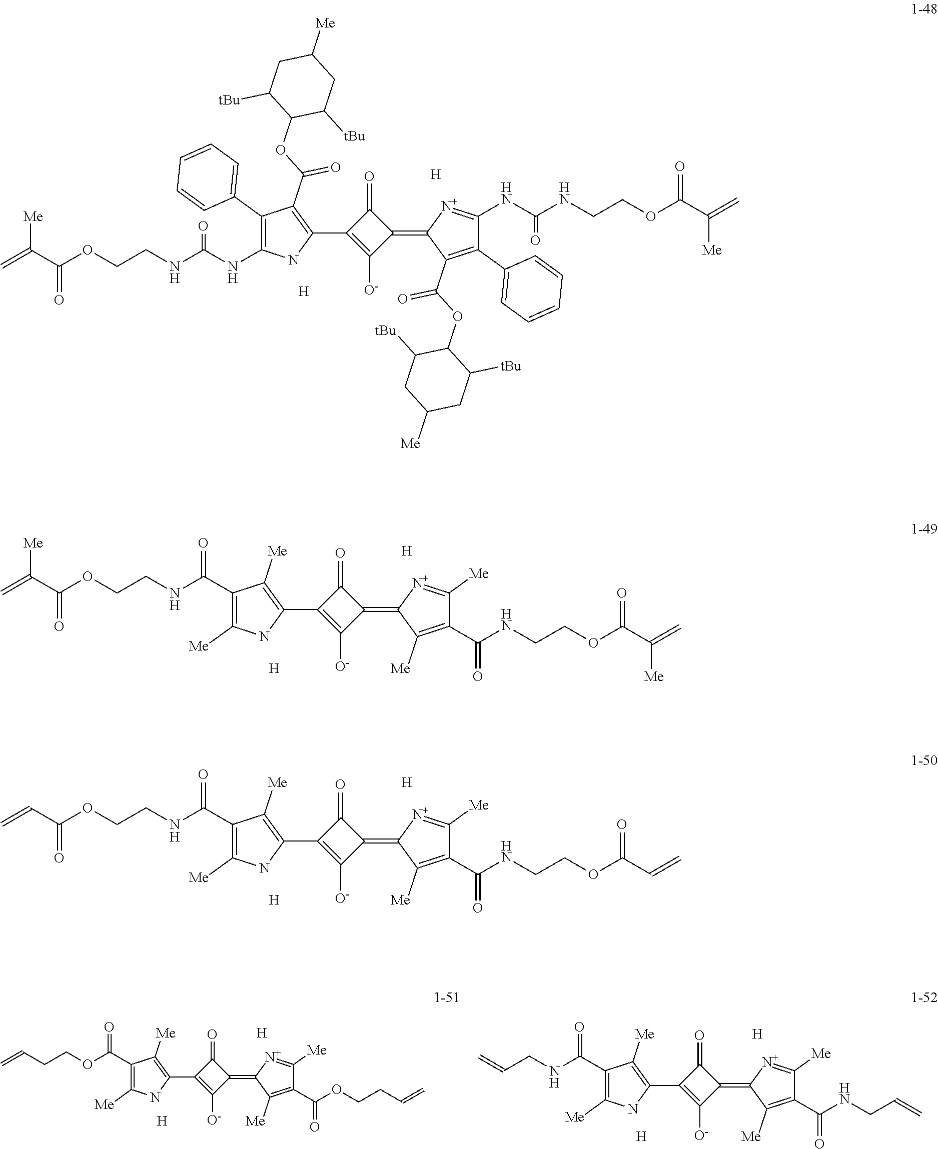

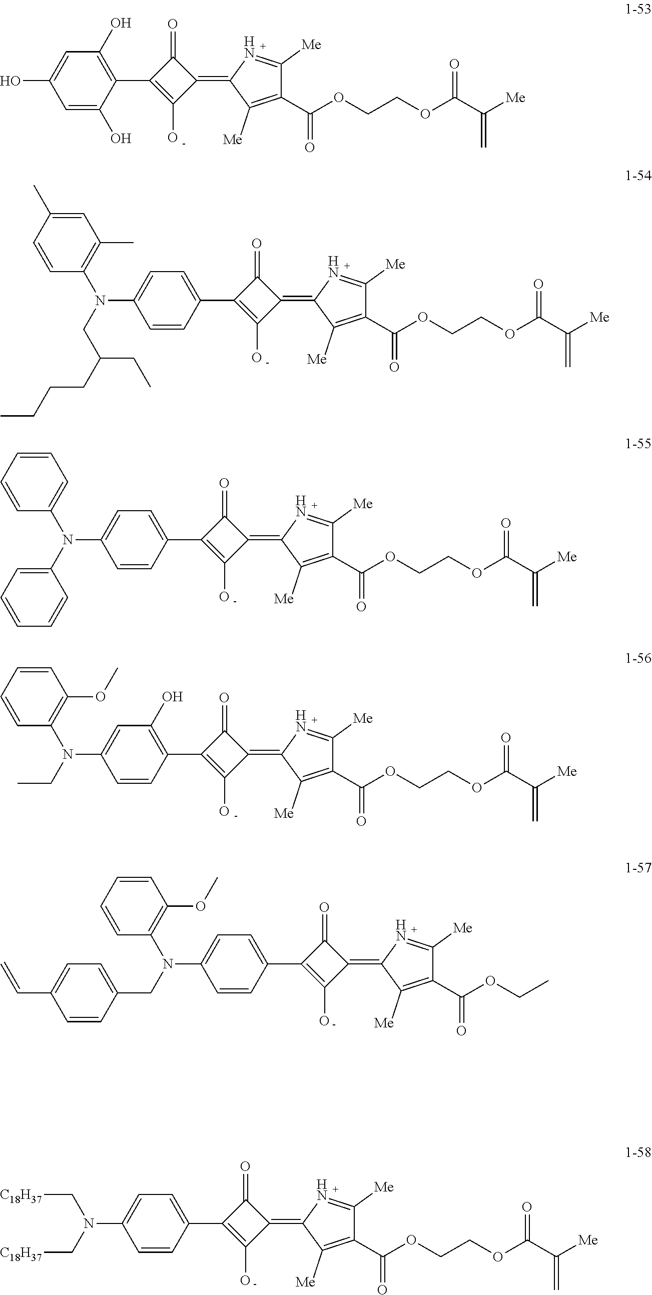

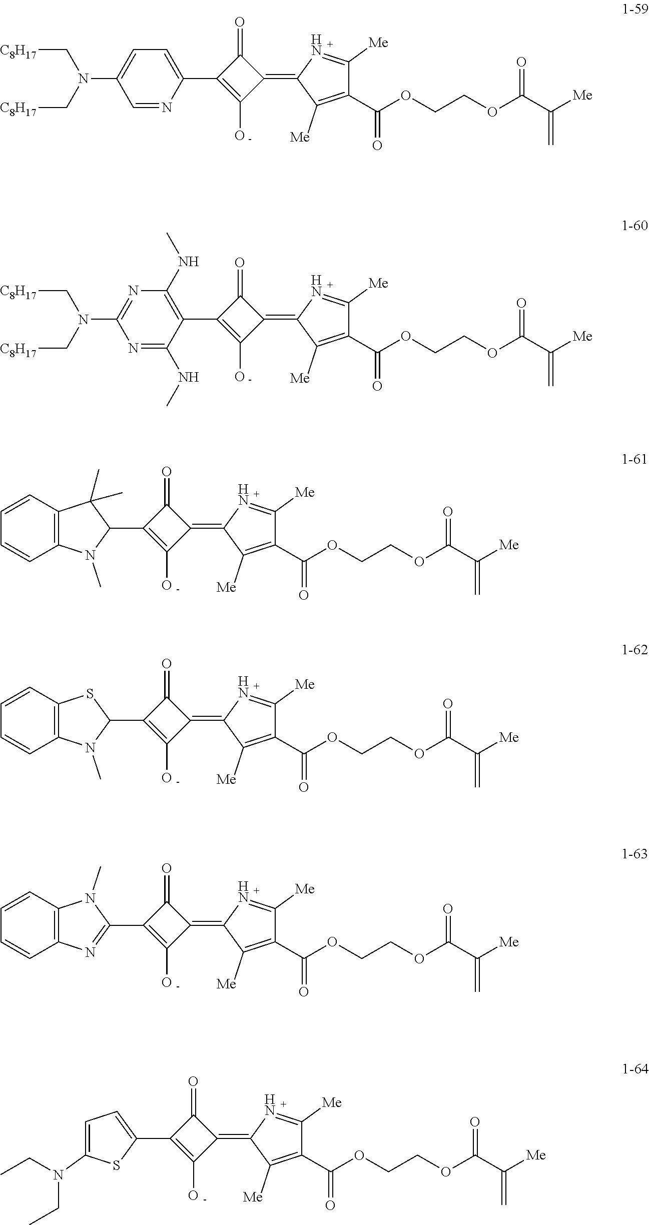

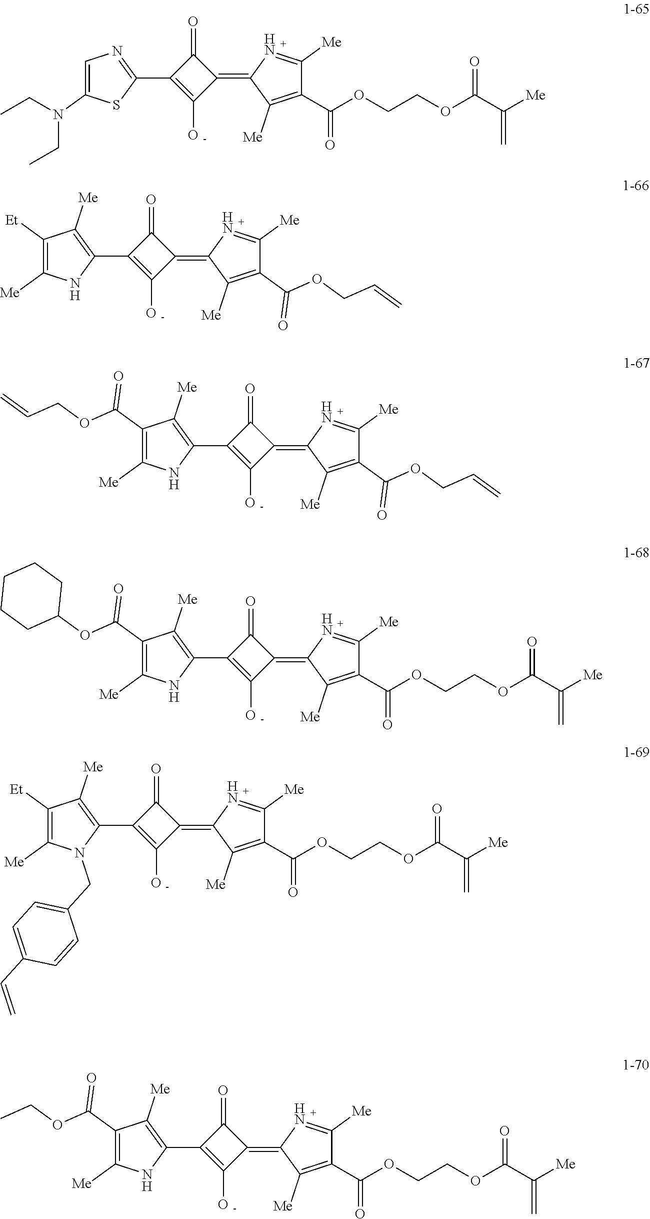

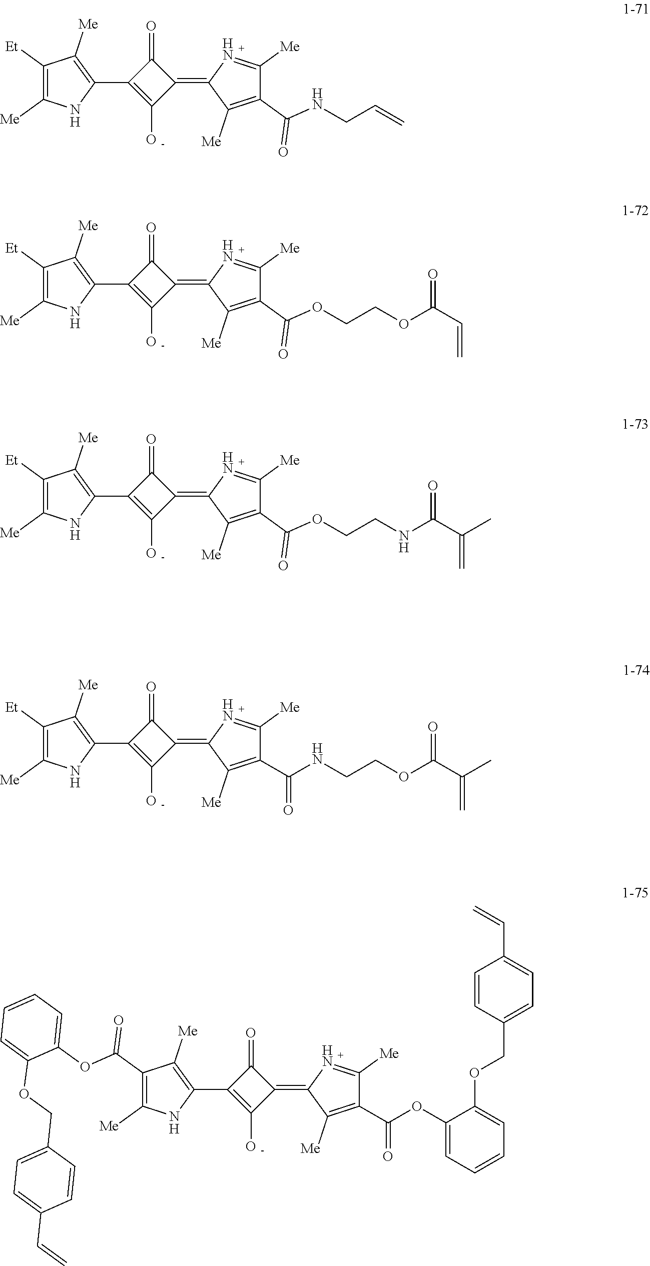

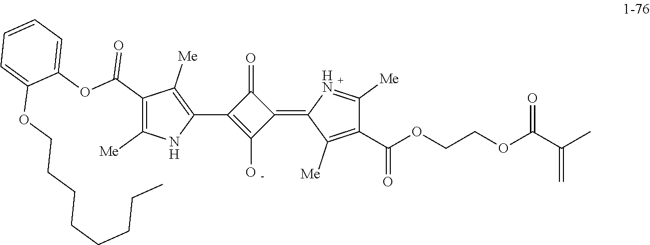

The maximal absorption wavelength of the compound according to the present disclosure is preferably in a range of 550 nm to 610 nm, and more preferably in a range of 555 nm to 605 nm.

A molar absorption coefficient of the compound according to the present disclosure at the maximal absorption wavelength is preferably 20,000 L/(mol·cm) or more, more preferably 50,000 L/(mol·cm) or more, and still more preferably 100,000 L/(mol·cm) or more.

In a visible absorption spectrum of the compound according to the present disclosure, a width of an absorption spectrum at an absorbance corresponding to ½ of the maximum absorbance (so-called a half-width) is preferably 60 nm or less, and more preferably 40 nm or less.

In addition, in the visible absorption spectrum of the compound according to the present disclosure, a width of an absorption spectrum at an absorbance corresponding to ⅛ of the maximum absorbance is preferably 100 nm or less, and more preferably 80 nm or less.

The above-described maximal absorption wavelength, molar absorption coefficient, and width of the absorption spectrum are measured using a spectrophotometer. As a specific measuring method thereof, methods described in Examples below can be referred to.

The compound according to the present disclosure has a characteristic of excellent solubility in an organic solvent and in a polymerizable compound described later. For example, regarding the compound according to the present disclosure, a solubility in ethyl acetate at 40° C. is preferably 0.1% by mass or more, and more preferably 1% by mass or more.

A molecular weight of the compound according to the present disclosure is not particularly limited, but for example, preferably 300 to 1,000 and more preferably 400 to 800.

### [Production Method of Compound]

The production method of the compound according to the present disclosure (hereinafter, also referred to as “production method according to the present disclosure”) is not particularly limited. The compound according to the present disclosure can be more suitably produced by the production method according to the present disclosure shown below.

Exemplary embodiments (first embodiment, second embodiment, and third embodiment) of the production method according to the present disclosure will be described.

### First Embodiment

The production method according to the first embodiment includes reacting a compound represented by Formula (4) with an aromatic compound in a solvent containing an alcohol.

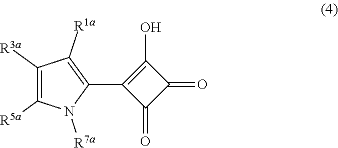

In Formula (4), R^{1a}, R^{3a}, R^{5a}, and R^{7a} each independently represent a hydrogen atom or a substituent, and R^{1a} and R^{3a}, R^{3a} and R^{5a}, and R^{5a} and R^{7a} each may be bonded to each other to form a ring, provided that at least one of R^{1a}, R^{3a}, R^{5a}, or R^{7a} represents a group which includes a polymerizable group having an ethylenically unsaturated bond.

R^{1a}, R^{3a}, R^{5a}, and R^{7a} in Formula (4) have the same meanings as R¹, R³, R⁵, and R⁷ in Formula (X), respectively, and preferred aspects thereof are also the same, so that the description thereof will be omitted here. From the viewpoint of productivity, it is preferable that at least one of R^{3a} or R^{7a} is the group which includes a polymerizable group having an ethylenically unsaturated bond.

The aromatic compound may be an aromatic hydrocarbon compound or a heteroaromatic compound.

The aromatic compound may be appropriately selected according to the structure of the desired compound represented by Formula (X).

In the production method according to the first embodiment, the compound represented by Formula (4) is reacted with the aromatic compound in a solvent containing an alcohol.

The solvent in which the compound represented by Formula (4) and the aromatic compound are reacted with each other contains an alcohol. The alcohol contained in the solvent may be only one kind or two or more kinds.

The alcohol is not particularly limited, but for example, preferably a monohydric alcohol, and more preferably at least one selected from the group consisting of methanol, ethanol, 1-propanol, 2-propanol, 1-butanol, 2-methylpropyl alcohol, 2-butanol, 1-pentanol, and 2-pentanol.

Among these, from the viewpoint of boiling point, the alcohol is preferably at least one selected from the group consisting of 1-butanol, 2-methylpropyl alcohol, 2-butanol, 1-pentanol, and 2-pentanol. In addition, for a compound which is likely to undergo a addition solvent decomposition, such as a compound having an alkoxycarbonyl group, it is preferable to use at least one selected from the group consisting of secondary alcohols, that is, 2-propanol, 2-butanol, and 2-pentanol.

The solvent in which the compound represented by Formula (4) and the aromatic compound are reacted with each other may contain only the alcohol, or may contain a solvent other than the alcohol (so-called other solvents).

As the other solvents, a hydrophobic solvent is preferable; at least one selected from the group consisting of an aliphatic hydrocarbon-based solvent and an aromatic hydrocarbon-based solvent is more preferable; and at least one selected from an aromatic hydrocarbon-based solvent is still more preferable.

Among the aromatic hydrocarbon-based solvents, as the other solvents, at least one selected from the group consisting of benzene, toluene, and xylene (for example, o-xylene, m-xylene, and p-xylene) is preferable; and toluene is more preferable.

A reaction ratio of the compound represented by Formula (4) and the aromatic compound can be appropriately set according to the structure of the desired compound represented by Formula (X).

For example, the reaction ratio of the compound represented by Formula (4) and the aromatic compound [Compound represented by Formula (4): Aromatic compound] is preferably 1.0:0.9 to 1.0:1.1.

A reaction temperature is not particularly limited, but for example, preferably room temperature (25° C.) to the boiling point of the reaction solvent, more preferably 60° C. to the boiling point of the reaction solvent, and still more preferably the boiling point of the reaction solvent.

A reaction time is not particularly limited, but for example, can be set to 1 hour to 24 hours.

The reaction between the compound represented by Formula (4) and the aromatic compound is preferably carried out under reflux of the organic solvent while performing azeotropic dehydration of water to be generated.

In the production method according to the first embodiment, for example, by controlling conditions such as the reaction ratio of the reaction between the compound represented by Formula (4) and the aromatic compound, and the reaction time, the compound represented by Formula (X), having the desired structure, can be produced, and production suitability is excellent.

### Second Embodiment

The production method according to the second embodiment is an example of a suitable production method in a case where the compound according to the present disclosure is the compound represented by Formula (1).

The production method according to the second embodiment includes reacting a compound represented by Formula (4) with a compound represented by Formula (5) in a solvent containing an alcohol.

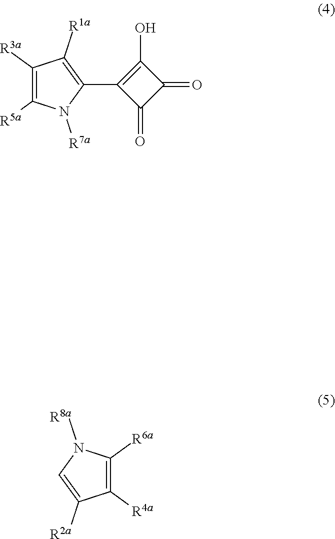

In Formula (4) and Formula (5), R^{1a}, R^{2a}, R^{3a}, R^{4a}, R^{5a}, R^{6a}, R^{7a}, and R^{8a} each independently represent a hydrogen atom or a substituent, and R^{1a} and R^{3a}, R^{2a} and R^{4a}, R^{3a} and R^{5a}, R^{4a} and R^{6a}, R^{5a} and R^{7a}, and R^{6a} and R^{8a} each may be bonded to each other to form a ring, provided that at least one of R^{1a}, R^{2a}, R^{3a}, R^{4a}, R^{5a}, R^{6a}, R^{7a}, or R^{8a} represents a group which includes a polymerizable group having an ethylenically unsaturated bond.

R^{1a}, R^{2a}, R^{3a}, R^{4a}, R^{5a}, R^{6a}, R^{7a}, and R^{8a} in Formula (4) and Formula (5) have the same meanings as R¹, R², R³, R⁴, R⁵, R⁶, R⁷, and R⁸ in Formula (1), respectively, and preferred aspects thereof are also the same, so that the description thereof will be omitted here.

From the viewpoint of productivity, it is preferable that at least one of R^{3a}, R^{4a}, R^{7a}, and R^{8a} is the group which includes a polymerizable group having an ethylenically unsaturated bond. In addition, from the same viewpoint, it is also preferable that at least one of R^{1a}, R^{3a}, R^{5a}, or R^{7a} and at least one of R^{2a}, R^{4a}, R^{6a}, or R^{8a} are the group which includes a polymerizable group having an ethylenically unsaturated bond.

In the production method according to the second embodiment, the compound represented by Formula (4) is reacted with the compound represented by Formula (5) in a solvent containing an alcohol.

The solvent in which the compound represented by Formula (4) and the compound represented by Formula (5) are reacted with each other contains an alcohol. The alcohol contained in the solvent may be only one kind or two or more kinds.

The alcohol in the production method according to the second embodiment has the same meaning as the alcohol in the production method according to the first embodiment, and preferred aspects thereof are also the same, so that the description thereof will be omitted here.

The solvent in which the compound represented by Formula (4) and the compound represented by Formula (5) are reacted with each other may contain only the alcohol, or may contain a solvent other than the alcohol (so-called other solvents).

The other solvents in the production method according to the second embodiment have the same meanings as the other solvents in the production method according to the first embodiment, and preferred aspects thereof are also the same, so that the description thereof will be omitted here.

A reaction ratio of the compound represented by Formula (4) and the compound represented by Formula (5) can be appropriately set according to the structure of the desired compound represented by Formula (1).

For example, the reaction ratio of the compound represented by Formula (4) and the compound represented by Formula (5) [Compound represented by Formula (4): Compound represented by Formula (5)] is preferably 1.0:0.9 to 1.0:1.1.

The reaction temperature and reaction time in the production method according to the second embodiment have the same meanings as the reaction temperature and reaction time in the production method according to the first embodiment, and preferred aspects thereof are also the same, so that the description thereof will be omitted here.

The reaction between the compound represented by Formula (4) and the compound represented by Formula (5) is preferably carried out under reflux of the organic solvent while performing azeotropic dehydration of water to be generated.

In the production method according to the second embodiment, for example, by controlling conditions such as the reaction ratio of the reaction between the compound represented by Formula (4) and the compound represented by Formula (5), and the reaction time, the compound represented by Formula (1), having the desired structure, can be produced, and production suitability is excellent.

### Third Embodiment

The production method according to the third embodiment is also an example of a suitable production method in a case where the compound according to the present disclosure is the compound represented by Formula (1).

The production method according to the third embodiment includes reacting a compound represented by Formula (6) with a compound represented by Formula (7) in a solvent containing an alcohol.

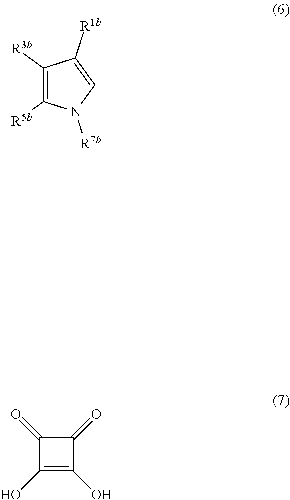

In Formula (6), R^{1b}, R^{3b}, R^{5b}, and R^{7b} each independently represent a hydrogen atom or a substituent, and R^{1b} and R^{3b}, R^{3b} and R^{5b}, and R^{5b} and R^{7b} each may be bonded to each other to form a ring, provided that at least one of R^{1b}, R^{3b}, R^{5b}, or R^{7b} represents a group which includes a polymerizable group having an ethylenically unsaturated bond.

R^{1b}, R^{3b}, R^{5b}, and R^{7b} in Formula (6) have the same meanings as R¹, R³, R⁵, and R⁷ in Formula (1), respectively, and preferred aspects thereof are also the same, so that the description thereof will be omitted here.

From the viewpoint of productivity, it is preferable that at least one of R^{3b} or R^{7b} is the group which includes a polymerizable group having an ethylenically unsaturated bond.

In the production method according to the third embodiment, the compound represented by Formula (6) is reacted with the compound represented by Formula (7) in a solvent containing an alcohol.

The solvent in which the compound represented by Formula (6) and the compound represented by Formula (7) are reacted with each other contains an alcohol. The alcohol contained in the solvent may be only one kind or two or more kinds.

The alcohol in the production method according to the third embodiment has the same meaning as the alcohol in the production method according to the first embodiment, and preferred aspects thereof are also the same, so that the description thereof will be omitted here.

The solvent in which the compound represented by Formula (6) and the compound represented by Formula (7) are reacted with each other may contain only the alcohol, or may contain a solvent other than the alcohol (so-called other solvents).

The other solvents in the production method according to the third embodiment have the same meanings as the other solvents in the production method according to the first embodiment, and preferred aspects thereof are also the same, so that the description thereof will be omitted here.

A reaction ratio of the compound represented by Formula (6) and the compound represented by Formula (7) can be appropriately set according to the structure of the desired compound represented by Formula (1).

The reaction temperature and reaction time in the production method according to the third embodiment have the same meanings as the reaction temperature and reaction time in the production method according to the first embodiment, and preferred aspects thereof are also the same, so that the description thereof will be omitted here.

The reaction between the compound represented by Formula (6) and the compound represented by Formula (7) is preferably carried out under reflux of the organic solvent while performing azeotropic dehydration of water to be generated.

In the production method according to the third embodiment, for example, by controlling conditions such as the reaction ratio of the reaction between the compound represented by Formula (6) and the compound represented by Formula (7), and the reaction time, the compound represented by Formula (1), having the desired structure, can be produced, and production suitability is excellent.

### <Applications>

The compound according to the present disclosure is preferably a compound used as a coloring agent.

The compound according to the present disclosure can be particularly suitably used as a dye.

Since the compound according to the present disclosure has a maximal absorption wavelength in the vicinity of 600 nm, the compound can be suitably used as a material for shielding light in a wavelength range in the vicinity of 600 nm. Here, the “shielding light in a wavelength range in the vicinity of 600 nm” means a case in which the light in the wavelength range in the vicinity of 600 nm is completely shielded and a case in which a part of the light in the wavelength range in the vicinity of 600 nm is shielded and a transmittance of the light in the wavelength range in the vicinity of 600 nm is decreased.

Specific examples of applications of the compound according to the present disclosure include an optical filter (for example, a color correction filter for a display) and a lens for glasses.

### [Polymerizable Composition]

The polymerizable composition according to the present disclosure contains the compound according to the present disclosure and a polymerizable compound.

With the polymerizable composition according to the present disclosure, in a case where a cured substance is formed by a polymerization reaction, the compound according to the present disclosure is fixed in the cured substance. Therefore, it is possible to obtain a cured substance in which the bleed-out of the compound according to the present disclosure is suppressed.

### [Compound According to Present Disclosure]

The polymerizable composition according to the present disclosure contains the compound according to the present disclosure.

The details of the compound according to the present disclosure are as described above, and thus the description thereof will be omitted.

The polymerizable composition according to the present disclosure may contain only one kind of the compound according to the present disclosure, or may contain two or more kinds thereof.

A content of the compound according to the present disclosure in the polymerizable composition according to the present disclosure is not particularly limited, and can be appropriately set according to the purpose.

The content of the compound according to the present disclosure in the polymerizable composition according to the present disclosure is, for example, preferably 0.01% by mass to 90% by mass with respect to the total solid content mass of the polymerizable composition. The lower limit thereof is more preferably 0.05% by mass or more and still more preferably 0.1% by mass or more. The upper limit thereof is more preferably 85% by mass or less and still more preferably 50% by mass or less.

### [Polymerizable Compound]

The polymerizable composition according to the present disclosure contains a polymerizable compound [excluding the compound according to the present disclosure].

The polymerizable compound may be any compound which can be polymerized and cured by application of energy, and is not particularly limited. As the polymerizable compound, a known polymerizable compound can be used.

The polymerizable compound is preferably a compound having a polymerizable group.

The polymerizable group is preferably a group having an ethylenically unsaturated bond.

Specific examples of the polymerizable group include a vinyl group, a (meth)allyl group, a (meth)acryloyl group, a (meth)acryloyloxy group, a (meth)acryloylamino group, and a vinylphenyl group.

The polymerizable compound is preferably a compound having one or more terminal ethylenically unsaturated bonds.

The polymerizable compound may be, for example, a monomer or a prepolymer (that is, a dimer, a trimer, or an oligomer), may be a mixture of the monomer and the prepolymer, may be a (co)polymer of the monomer, may be a (co)polymer of the prepolymer, or may be a copolymer of the monomer and the prepolymer.

Examples of the monomer include an unsaturated carboxylic acid, an ester of an unsaturated carboxylic acid, and an amide of an unsaturated carboxylic acid.

Specific examples of the unsaturated carboxylic acid include (meth)acrylic acid, itaconic acid, crotonic acid, isocrotonic acid, and maleic acid.

It is preferable that the polymerizable compound is at least one selected from the group consisting of a (meth)acrylic acid, a (meth)acrylic acid amide compound, a (meth)acrylic acid ester compound, and a styrene compound.

Specific examples of the (meth)acrylic acid amide compound include (meth)acrylic acid amide, N,N-dimethyl acrylamide, N-isopropyl acrylamide, methylenebis (acrylamide), 2-acrylamido-2-methylpropanesulfonic acid, and N-(3-dimethylaminopropyl) methacrylamide.

Specific examples of the (meth)acrylic acid ester compound include methyl (meth)acrylate, ethyl (meth)acrylate, n-butyl (meth)acrylate, 2-hydroxyethyl (meth)acrylate, glycidyl (meth)acrylate, benzyl (meth)acrylate, 2-(2-phenoxy) ethyl (meth)acrylate, 2-ethylhexyl (meth)acrylate, n-octyl (meth)acrylate, isooctyl (meth)acrylate, n-nonyl (meth)acrylate, isononyl (meth)acrylate, n-decyl (meth)acrylate, isodecyl (meth)acrylate, n-dodecyl (meth)acrylate, n-tridecyl (meth)acrylate, n-tetradecyl (meth)acrylate, n-hexadecyl (meth)acrylate, stearyl (meth)acrylate, isobornyl (meth)acrylate, dicyclopentenyl (meth)acrylate, dicyclopentanyl (meth)acrylate, dicyclopentenyloxyethyl (meth)acrylate, 1-hydroxyheptyl (meth)acrylate, 1-hydroxybutyl (meth)acrylate, 1-hydroxypentyl (meth)acrylate, 2-hydroxybutyl (meth)acrylate, ethylene glycol diacrylate, ethylene glycol dimethacrylate, polyethylene glycol diacrylate, polyethylene glycol dimethacrylate, ethoxylated bisphenol A diacrylate, ethoxylated bisphenol A dimethacrylate, ethoxylated trimethylolpropane triacrylate, ethoxylated trimethylolpropane trimethacrylate, ethoxylated glycerin triacrylate, ethoxylated glycerin trimethacrylate, ethoxylated pentaerythritol tetraacrylate, ethoxylated pentaerythritol tetramethacrylate, ethoxylated dipentaerythritol hexaacrylate, polyglycerin monoethylene oxide polyacrylate, polyglycerin polyethylene glycol polyacrylate, dipentaerythritol hexaacrylate, dipentaerythritol hexamethacrylate, neopentyl glycol diacrylate, neopentyl glycol dimethacrylate, pentaerythritol triacrylate, pentaerythritol trimethacrylate, trimethylolpropane triacrylate, trimethylolpropane trimethacrylate, tricyclodecane dimethanol diacrylate, tricyclodecane dimethanol dimethacrylate, 1,6-hexanediol diacrylate, and 1,6-hexanediol dimethacrylate.

Specific examples of the styrene compound include styrene, methylstyrene, dimethylstyrene, trimethylstyrene, ethylstyrene, fluorostyrene, chlorostyrene, methoxystyrene, tert-butoxystyrene, and divinylbenzene.

Regarding the polymerizable compound, details of the usage method, such as what kind of polymerizable compound having what kind of structure is used, whether the polymerizable compound is used alone or in combination of two or more kinds thereof, and how much the content thereof is set, can be optionally set according to the final performance design of the polymerizable composition.

For example, from the viewpoint of sensitivity, as the polymerizable compound, a compound having a structure in which the amount of polymerizable groups per molecule is large is preferable, and in many cases, a bi- or higher functional compound is preferable.

For example, from the viewpoint of film hardness, as the polymerizable compound, a tri- or higher functional compound [for example, a hexafunctional (meth)acrylic acid ester compound] may be used.

For example, it is also effective to select a polymerizable compound in consideration of compatibility, dispersibility, and the like with each component contained in the composition.

As the polymerizable compound, compounds having different functionalities and/or different polymerizable groups [for example, the (meth)acrylic acid ester compound, the styrene compound, and the vinyl ether compound] may be used in combination.

A molecular weight of the polymerizable compound is not particularly limited, but for example, preferably 100 to 3,000, more preferably 100 to 2,600, and still more preferably 100 to 2,200.

The polymerizable composition according to the present disclosure may contain only one kind of the polymerizable compound, or may contain two or more kinds thereof.

A content of the polymerizable compound in the polymerizable composition according to the present disclosure is not particularly limited, but for example, preferably 30% by mass or more and less than 100% by mass, more preferably 50% by mass or more and less than 100% by mass, and still more preferably 60% by mass or more and less than 100% by mass with respect to the total solid content mass of the polymerizable composition.

### [Polymerization Initiator]

The polymerizable composition according to the present disclosure can contain a polymerization initiator.

The polymerization initiator may be any compound capable of generating an initiating species required for the polymerization reaction by application of energy, and is not particularly limited. As the polymerization initiator, a known polymerization initiator can be used.

Examples of the polymerization initiator include a photopolymerization initiator and a thermal polymerization initiator.

As the photopolymerization initiator, an activator having photosensitivity to light in a range from an ultraviolet ray range to a visible range is preferable. In addition, the photopolymerization initiator may be an activator which produces an active radical by causing some action with a photoexcited sensitizer.

Examples of the photopolymerization initiator include a halogenated hydrocarbon derivative (for example, a compound having a triazine skeleton and a compound having an oxadiazole skeleton), an acylphosphine compound, hexaaryl biimidazole, an oxime compound (for example, an oxime ester compound), an organic peroxide, a thio compound, a ketone compound, an aromatic onium salt, an aminoacetophenone compound, and a hydroxyacetophenone compound.

Examples of the acylphosphine compound include acylphosphine-based initiators described in JP4225898B.

Examples of the oxime compound include compounds described in JP2001-233842A, compounds described in JP2000-080068A, compounds described in JP2006-342166A, and

compounds described in paragraphs to of JP2016-006475A. Among the oxime compounds, an oxime ester compound is preferable.

Examples of the aminoacetophenone compound include compounds described in JP2009-191179A and aminoacetophenone-based initiators described in JP1998-291969A (JP-H10-291969A).

Examples of the thermal polymerization initiator include an azo-based compound, an organic peroxide, and an inorganic peroxide.

Specific examples of the azo-based compound include dimethyl 2,2′-azobis (isobutyrate), 2,2′-azobisisobutyronitrile, 2,2′-azobis (2,4-dimethyl-4-methoxyvaleronitrile), 2,2′-azobis (2,4-dimethylvaleronitrile), dimethyl-2,2′-azobis (2-methylpropionate), 2,2′-azobis (2-methylbutyronitrile), 2,2′-azobis (N-butyl-2-methylpropionamide), 1,1′-azobis (cyclohexane-1-carbonitrile), dimethyl-1,1′-azobis (1-cyclohexanecarboxylate), and 2,2′-azobis [2-(2-imidazolin-2-yl)propane]dihydrochloride.

Specific examples of the organic peroxide include 1,1-di(tert-hexylperoxy)cyclohexane, 1,1-di(tert-butylperoxy)cyclohexane, 2,2-di (4,4-di-(tert-butylperoxy) cyclohexyl)propane, tert-hexyl peroxy isopropyl monocarbonate, tert-butyl peroxy-3,5,5-trimethyl hexanoate, tert-butyl peroxy laurate, dicumyl peroxide, di-tert-butyl peroxide, tert-butyl peroxy-2-ethyl hexanoate, tert-hexyl peroxy-2-ethyl hexanoate, cumene hydroperoxide, and tert-butyl hydroperoxide.

Specific examples of the inorganic peroxide include potassium persulfate, ammonium persulfate, and hydrogen peroxide.

The polymerization initiator may be a synthetic product or a commercially available product.

Examples of the commercially available product of the photopolymerization initiator include IRGACURE (registered trademark) OXE01 [manufactured by BASF SE], TR-PBG-304 [manufactured by Changzhou Tronly New Electronic Materials Co., Ltd.], and ADEKA ARKLS (registered trademark) NCI-831 and ADEKA ARKLS (registered trademark) NCI-930 [both manufactured by ADEKA Corporation].

In addition, examples of the commercially available product of the photopolymerization initiator, which is the hydroxyacetophenone compound, include Omnirad (registered trademark) 184, Omnirad (registered trademark) 1173, Omnirad (registered trademark) 2959, and Omnirad (registered trademark) 127 [all manufactured by IGM Resins B.V.].

Examples of the commercially available product of the photopolymerization initiator, which is the aminoacetophenone compound, include Omnirad (registered trademark) 907, Omnirad (registered trademark) 369, Omnirad (registered trademark) 369E, and Omnirad (registered trademark) 379EG [all manufactured by IGM Resins B.V.].

Examples of the commercially available product of the photopolymerization initiator, which is the acylphosphine compound, include Omnirad (registered trademark) 819 and Omnirad (registered trademark) TPO [both manufactured by IGM Resins B.V.].

Examples of the commercially available product of the photopolymerization initiator, which is the oxime compound, include Irgacure (registered trademark) OXE01, Irgacure (registered trademark) OXE02, and Irgacure (registered trademark) OXE03 [all manufactured by BASF SE].

In a case where the polymerizable composition according to the present disclosure contains a polymerization initiator, the polymerizable composition according to the present disclosure may contain only one kind of the polymerization initiator, or may contain two or more kinds thereof.

In a case where the polymerizable composition according to the present disclosure contains a polymerization initiator, a content of the polymerization initiator is not particularly limited, but for example, preferably 0.1% by mass to 20% by mass, more preferably 0.3% by mass to 15% by mass, and still more preferably 0.4% by mass to 10% by mass with respect to the total solid content mass of the polymerizable composition.

### [Ultraviolet Absorber]

The polymerizable composition according to the present disclosure may further contain an ultraviolet absorber.

The ultraviolet absorber is not particularly limited, and examples thereof include an aminobutadiene-based compound, a dibenzoylmethane-based compound, a benzotriazole-based compound, a benzophenone-based compound, and a hydroxyphenyltriazine-based compound.

Among these, as the ultraviolet absorber, at least one selected from the group consisting of a benzotriazole-based compound, a benzophenone-based compound, and a hydroxyphenyltriazine-based compound is preferable.

In the present disclosure, the “aminobutadiene-based compound” refers to a “compound having an aminobutadiene skeleton in the molecule”; the “dibenzoylmethane-based compound” refers to a “compound having a dibenzoylmethane skeleton in the molecule”; the “benzotriazole-based compound” refers to a “compound having a benzotriazole skeleton in the molecule”; the “benzophenone-based compound” refers to a “compound having a benzophenone skeleton in the molecule”; and the “hydroxyphenyltriazine-based compound” refers to a “compound having a hydroxyphenyltriazine skeleton in the molecule”.

The ultraviolet absorber may or may not have a polymerizable group in the molecule.

Specific examples of the polymerizable group include a vinyl group, a (meth)allyl group, a (meth)acryloyl group, a (meth)acryloyloxy group, a (meth)acryloylamino group, and a vinylphenyl group.

As the ultraviolet absorber, for example, compounds described JP2003-128730A, JP2003-129033A, JP2014-077076A, JP2015-164994A, JP2015-168822A, JP2018-135282A, JP2018-168089A, JP2018-168278A, JP2018-188589A, JP2019-001767A, JP2020-023697A, JP2020-041013A, JP5518613B, JP5868465B, JP6301526B, JP6354665B, JP2017-503905A, WO2015/064674A, WO2015/064675A, WO2017/102675A, WO2017/122503A, WO2018/190281A, WO2018/216750A, WO2019/087983A, WO2021/029146A, EP2379512B, and EP2951163B can be used.

Examples of a commercially available product of the ultraviolet absorber include Tinuvin (registered trademark) P, Tinuvin (registered trademark) 234, Tinuvin (registered trademark) 326, Tinuvin (registered trademark) 571, and Tinuvin (registered trademark) 970 [all manufactured by BASF SE], which are benzotriazole-based compounds; and Tinuvin (registered trademark) 1577 and Tinuvin (registered trademark) 1600 [both manufactured by BASF SE], which are triazine compounds. In addition, examples of the commercially available product of the ultraviolet absorber include RUVA-93 [product name, component: 2-[2-hydroxy-5-(2-methacryloyloxyethyl) phenyl]2H-benzo[d][1,2,3]triazole, manufactured by Otsuka Chemical Co.,Ltd.].

In a case where the polymerizable composition according to the present disclosure contains an ultraviolet absorber, the polymerizable composition according to the present disclosure may contain only one kind of the ultraviolet absorber, or may contain two or more kinds thereof.

In a case where the polymerizable composition according to the present disclosure contains an ultraviolet absorber, a content of the ultraviolet absorber is not particularly limited, but for example, preferably 0.01% by mass to 10% by mass and more preferably 0.01% by mass to 5% by mass with respect to the total solid content mass of the polymerizable composition.

### [Polymer Compound]

The polymerizable composition according to the present disclosure may further contain a polymer compound.

The polymer compound is not particularly limited, but for example, a resin is preferable.

The resin is not particularly limited, and examples thereof include a (meth)acrylic resin, a polyester resin, a polycarbonate resin, a vinyl polymer (for example, a polydiene resin, a polyalkene resin, a polystyrene resin, a polyvinyl ether resin, a polyvinyl alcohol resin, a polyvinyl ketone resin, a polyfluorovinyl resin, and a polyvinyl bromide resin), a polythioether resin, a polyphenylene resin, a polyurethane resin, a polysulfonate resin, a nitroso polymer resin, a polysiloxane resin, a polysulfide resin, a polythioester resin, a polysulfone resin, a polysulfonamide resin, a polyamide resin, a polyimine resin, a polyurea resin, a polyphosphazene resin, a polysilane resin, a polysilazane resin, a polyfuran resin, a polybenzoxazole resin, a polyoxadiazole resin, a polybenzothiazinophenothiazine resin, a polybenzothiazole resin, a polypyrazinoquinoxaline resin, a polypyromellitimide resin, a polyquinoxaline resin, a polybenzoimidazoline resin, a polyoxoisoindoline resin, a polydioxoisoindoline resin, a polytriazine resin, a polypyridazine resin, a polypiperazine resin, a polypyridine resin, a polypiperidine resin, a polytriazole resin, a polypyrazole resin, a polypyrrolidine resin, a polycarborane resin, a polyoxabicyclononane resin, a polydibenzofuran resin, a polyphthalide resin, a polyacetal resin, a polyimide resin, an olefin resin, a cyclic olefin resin, an epoxy resin, and a cellulose acylate resin.

With regard to the details of the resin, reference can be made to, for example, description in paragraphs to of JP2009-263616A, the contents of which are incorporated herein by reference.

From the viewpoint that the resin has favorable compatibility with the compound represented by Formula (1) and a film in which surface unevenness is suppressed is easily obtained in a case of forming the film, the resin is preferably at least one resin selected from the group consisting of a (meth)acrylic resin, a polyester resin, a polystyrene resin, a polyurethane resin, a polycarbonate resin, an epoxy resin, and a cellulose acylate resin; and more preferably at least one resin selected from the group consisting of a (meth)acrylic resin, a polyester resin, a polystyrene resin, a polyurethane resin, an epoxy resin, and a cellulose acylate resin.

Examples of a commercially available product of the (meth)acrylic resin include SK Dyne series [product example: SK Dyne (registered trademark) SF2147] of Soken Chemical & Engineering Co., Ltd.

Examples of a commercially available product of the polyester resin include VYLON series [product example: VYLON 500] of Toyobo Co., Ltd.

The polystyrene resin is a resin in which a repeating unit derived from a styrene compound (also referred to as “styrene-based monomer”) is included in an amount of preferably 50% by mass or more, more preferably 70% by mass or more, and still more preferably 85% by mass or more with respect to all repeating units.

Specific examples of the styrene-based monomer include styrene and styrene derivatives. Here, the “styrene derivative” refers to a compound in which another group is bonded to styrene.

Specific examples of the styrene derivative include alkylstyrene (for example, o-methylstyrene, m-methylstyrene, p-methylstyrene, 2,4-dimethylstyrene, o-ethylstyrene, and p-ethylstyrene). In addition, specific examples of the styrene derivative include a substituted styrene in which a hydroxy group, an alkoxy group, a carboxyl group, a halogen, or the like is introduced into a benzene nucleus of styrene (for example, hydroxystyrene, tert-butoxystyrene, vinyl benzoic acid, o-chlorostyrene, and p-chlorostyrene).

The polystyrene resin may include a repeating unit derived from a monomer other than the styrene-based monomer. Examples of other monomers include an alkyl (meth)acrylate, an unsaturated carboxylic acid monomer, an unsaturated dicarboxylic acid anhydride monomer, an unsaturated nitrile monomer, and a conjugated diene.

Specific examples of the alkyl (meth)acrylate include methyl (meth)acrylate, cyclohexyl (meth)acrylate, methylphenyl (meth)acrylate, and isopropyl (meth)acrylate.

Specific examples of the unsaturated carboxylic acid monomer include methacrylic acid, acrylic acid, itaconic acid, maleic acid, fumaric acid, and cinnamic acid.

Specific examples of the unsaturated dicarboxylic acid anhydride monomer include maleic acid anhydride and anhydrides of itaconic acid, ethyl maleic acid, methyl itaconic acid, and chloromaleic acid.

Specific examples of the unsaturated nitrile monomer include acrylonitrile and methacrylonitrile.

Specific examples of the conjugated diene include 1,3-butadiene, 2-methyl-1,3-butadiene (isoprene), 2,3-dimethyl-1,3-butadiene, 1,3-pentadiene, and 1,3-hexadiene.

Examples of a commercially available product of the polystyrene resin include AS-70 [acrylonitrile/styrene copolymer resin, manufactured by NIPPON STEEL Chemical & Material Co., Ltd.] and SMA2000P [styrene/maleic acid copolymer, manufactured by Kawahara Petrochemical Co., Ltd.].

Examples of the epoxy resin include a bisphenol A-type epoxy resin, a bisphenol F-type epoxy resin, a phenol novolac-type epoxy resin, a cresol novolac-type epoxy resin, and an aliphatic epoxy resin.

Examples of a commercially available product of the bisphenol A-type epoxy resin include JER827, JER828, JER834, JER1001, JER1002, JER1003, JER1055, JER1007, JER1009, and JER1010 [all manufactured by Mitsubishi Chemical Corporation]; and EPICLON (registered trademark) 860, EPICLON (registered trademark) 1050, EPICLON (registered trademark) 1051, and EPICLON (registered trademark) 1055 [all manufactured by DIC Corporation].

Examples of a commercially available product of the bisphenol F-type epoxy resin include JER806, JER807, JER4004, JER4005, JER4007, and JER4010 [all manufactured by Mitsubishi Chemical Corporation]; EPICLON (registered trademark) 830 and EPICLON (registered trademark) 835 [both manufactured by DIC Corporation]; and LCE-21 and RE-602S [both manufactured by Nippon Kayaku Co., Ltd.].

Examples of a commercially available product of the phenol novolac-type epoxy resin include JER152, JER154, JER157S70, and JER157S65 [all manufactured by Mitsubishi Chemical Corporation]; and EPICLON (registered trademark)N-740, EPICLON (registered trademark)N-770, and EPICLON (registered trademark)N-775 [all manufactured by DIC Corporation].

Examples of a commercially available product of the cresol novolac-type epoxy resin include EPICLON (registered trademark)N-660, EPICLON (registered trademark)N-665, EPICLON (registered trademark)N-670, EPICLON (registered trademark)N-673, EPICLON (registered trademark)N-680, EPICLON (registered trademark)N-690, and EPICLON (registered trademark)N-695 [all manufactured by DIC Corporation]; and EOCN-1020 [manufactured by Nippon Kayaku Co., Ltd.].

The cellulose acylate resin is, for example, preferably cellulose acylate described in paragraphs to of JP2012-215689A.

The resin is preferably an alkali-soluble resin.

In the present disclosure, the “alkali-soluble” refers to being soluble in a 1 mol/L sodium hydroxide solution at 25° C. In addition, the “soluble” refers to being dissolved in an amount of 0.1 g or more in 100 mL of a solvent.

The alkali-soluble resin is preferably a resin having a group which promotes alkali solubility (hereinafter, also referred to as “acid group”).

Examples of the acid group include a carboxy group, a phosphoric acid group, a sulfonic acid group, and a phenolic hydroxy group.

Among these, as the acid group, a carboxy group is preferable.

In a case where the resin has an acid group, the resin may have only one kind of the acid group or may have two or more kinds thereof.

The alkali-soluble resin is preferably a polymer having a carboxy group in a side chain.

Examples of the alkali-soluble resin include (meth)acrylic acid copolymers, itaconic acid copolymers, crotonic acid copolymers, maleic acid copolymers, partially esterified maleic acid copolymers, alkali-soluble phenol resins such as a novolac-type resin, acidic cellulose derivatives having a carboxy group in the side chain, and resins in which an acid anhydride is added to a polymer having a hydroxy group.

The alkali-soluble resin is particularly preferably a copolymer of (meth)acrylic acid and other monomers copolymerizable with the (meth)acrylic acid [that is, a (meth)acrylic acid copolymer].

Examples of the other monomers copolymerizable with the (meth)acrylic acid include alkyl (meth)acrylate, aryl (meth)acrylate, and a vinyl compound.

Specific examples of the other monomers copolymerizable with the (meth)acrylic acid include methyl (meth)acrylate, ethyl (meth)acrylate, propyl (meth)acrylate, butyl (meth)acrylate, isobutyl (meth)acrylate, pentyl (meth)acrylate, hexyl (meth)acrylate, octyl (meth)acrylate, phenyl (meth)acrylate, benzyl (meth)acrylate, tolyl (meth)acrylate, naphthyl (meth)acrylate, cyclohexyl (meth)acrylate, styrene, α-methylstyrene, vinyl toluene, glycidyl methacrylate, acrylonitrile, vinyl acetate, N-vinylpyrrolidone, tetrahydrofurfuryl methacrylate, a polystyrene macromonomer, and a polymethyl methacrylate macromonomer.

In addition, examples of the other monomers copolymerizable with the (meth)acrylic acid include N-position-substituted maleimides described in JP1998-300922A (JP-H10-300922A) (for example, N-phenylmaleimide and N-cyclohexylmaleimide).

In the (meth)acrylic acid copolymer, the other monomers copolymerizable with the (meth)acrylic acid may be only one kind or may be two or more kinds.

In addition, preferred examples of the alkali-soluble resin also include a benzyl (meth)acrylate/(meth)acrylic acid copolymer, a benzyl (meth)acrylate/(meth)acrylic acid/2-hydroxyethyl (meth)acrylate copolymer, and a multicomponent copolymer formed of benzyl (meth)acrylate/(meth)acrylic acid/other monomers.

In addition, preferred examples of the alkali-soluble resin also include resins obtained by copolymerizing 2-hydroxyethyl (meth)acrylate, and a copolymer of 2-hydroxypropyl (meth)acrylate/polystyrene macromonomer/benzyl methacrylate/methacrylic acid, a copolymer of 2-hydroxy-3-phenoxypropyl acrylate/polymethyl methacrylate macromonomer/benzyl methacrylate/methacrylic acid, a copolymer of 2-hydroxyethyl methacrylate/polystyrene macromonomer/methyl methacrylate/methacrylic acid, and a copolymer of 2-hydroxyethyl methacrylate/polystyrene macromonomer/benzyl methacrylate/methacrylic acid, which are described in JP1995-140654A (JP-H7-140654A).

With regard to the details of the alkali-soluble resin, reference can be made to, for example, description in JP2012-208494A and JP2012-198408A, the contents of which are incorporated herein by reference.

An acid value of the alkali-soluble resin is not particularly limited, but for example, preferably 30 mgKOH/g to 200 mgKOH/g. The lower limit thereof is more preferably 50 mgKOH/g or more, and still more preferably 70 mgKOH/g or more. The upper limit thereof is more preferably 150 mgKOH/g or less, and still more preferably 120 mgKOH/g or less.

In the present disclosure, the acid value is a value measured by a method described in JIS K0070: 1992.

The resin may have a polymerizable group.

In a case where the polymerizable composition according to the present disclosure contains a resin having a polymerizable group, a film having higher hardness can be formed.

Specific examples of the polymerizable group include a vinyl group, a (meth)allyl group, a (meth)acryloyl group, a (meth)acryloyloxy group, a (meth)acryloylamino group, and a vinylphenyl group.

Examples of a commercially available product of the resin having a polymerizable group include DIANAL BR Series [type of resin: polymethyl methacrylate (PMMA), product examples: DIANAL (registered trademark) BR-80, BR-83, and BR-87, manufactured by Mitsubishi Chemical Corporation], Photomer 6173 [type of resin: COOH-containing polyurethane acrylic oligomer, manufactured by Diamond Shamrock Co., Ltd.], VISCOAT R-264 and KS RESIST 106 [both manufactured by Osaka Organic Chemical Industry Ltd.], CYCLOMER P Series [product example: CYCLOMER (registered trademark) P (ACA) Z230AA, manufactured by Daicel Corporation] and PLACCEL CF200 Series [manufactured by Daicel Corporation], EBECRYL (registered trademark) 3800 [manufactured by Daicel-Allnex Ltd.], and ACRYCURE (registered trademark) RD-F8 [manufactured by Nippon Shokubai Co., Ltd.].

A weight-average molecular weight of the resin is not particularly limited.

A weight-average molecular weight of the epoxy resin is, for example, preferably 100 or more, and more preferably 200 or more. In addition, the weight-average molecular weight of the epoxy resin is, for example, preferably 2,000,000 or less, more preferably 1,000,000 or less, and still more preferably 500,000 or less.

A weight-average molecular weight of the resin other than the epoxy resin is, for example, preferably 3,000 or more, more preferably 4,000 or more, and still more preferably 5,000 or more. In addition, the weight-average molecular weight of the resin other than the epoxy resin is, for example, preferably 2,000,000 or less, more preferably 1,000,000 or less, and still more preferably 500,000 or less.

The total light transmittance of the resin is preferably 80% or more, more preferably 85% or more, and still more preferably 90% or more. In the present disclosure, the total light transmittance of the resin is a value measured based on the contents described in “The Fourth Series of Experimental Chemistry 29 Polymer Material” (Maruzen, 1992), pp. 225 to 232, edited by the Chemical Society of Japan.

In a case where the polymerizable composition according to the present disclosure contains a polymer compound (preferably, a resin; the same applies hereinafter), the polymerizable composition according to the present disclosure may contain only one kind of the polymer compound, or may contain two or more kinds thereof.

In a case where the polymerizable composition according to the present disclosure contains a polymer compound, a content of the polymer compound is not particularly limited, but for example, preferably 1% by mass to 99.9% by mass with respect to the total solid content mass of the polymerizable composition. The lower limit thereof is more preferably 5% by mass or more and still more preferably 7% by mass or more. The upper limit thereof is more preferably 99% by mass or less and still more preferably 95% by mass or less.

### [Solvent]

The polymerizable composition according to the present disclosure may contain a solvent.

In the polymerizable composition according to the present disclosure, the solvent can contribute to, for example, adjustment of viscosity.

With the solvent, for example, the viscosity of the polymerizable composition can be adjusted to a suitable viscosity as a coating liquid. In a case where the polymerizable composition according to the present disclosure contains a low-molecular-weight monomer as the above-described polymerizable compound, the monomer may function as the solvent. In this case, the polymerizable composition according to the present disclosure may not contain the solvent, but may further contain a solvent for adjusting physical properties or the like.

The solvent is not particularly limited and can be appropriately selected, for example, in consideration of coating properties of the polymerizable composition according to the present disclosure and solubility of each component contained in the polymerizable composition according to the present disclosure.

For example, it is preferable that the solvent is selected in consideration of not only the solubility or dispersibility of the compound according to the present disclosure and the polymerizable compound, but also the solubility or dispersibility of components contained in the polymerizable composition as desired [for example, the polymerization initiator, the polymer compound, the ultraviolet absorber, the surfactant, a colorant, and the like], the coating surface state in a case of coating, the case of handling, and the like.

Examples of the solvent include water and an organic solvent.

The solvent is preferably an organic solvent.

Since the compound according to the present disclosure contained in the polymerizable composition according to the present disclosure has excellent solubility in the organic solvent, in a case where the solvent is an organic solvent, the bleed-out is less likely to occur.

The organic solvent is not particularly limited, and examples thereof include an ester compound, an ether compound, a ketone compound, and an aromatic hydrocarbon compound.

Examples of the ester compound include ethyl acetate, n-butyl acetate, isobutyl acetate, amyl formate, isoamyl acetate, isobutyl acetate, butyl propionate, isopropyl butyrate, ethyl butyrate, butyl butyrate, methyl lactate, ethyl lactate, an oxyacetic acid alkyl ester compound, a 3-oxypropionic acid alkyl ester compound, a 2-oxypropionic acid alkyl ester compound, methyl 2-oxy-2-methyl propionate (for example, methyl 2-methoxy-2-methyl propionate), ethyl 2-oxy-2-methyl propionate (for example, ethyl 2-ethoxy-2-methyl propionate), methyl pyruvate, ethyl pyruvate, propyl pyruvate, methyl acetoacetate, ethyl acetoacetate, methyl 2-oxobutanoate, ethyl 2-oxobutanoate, cyclohexyl acetate, and 1-methyl-2-methoxyethyl propionate.

Examples of the oxyacetic acid alkyl ester compound include methyl oxoacetate, ethyl oxoacetate, and butyl oxoacetate, and specific examples thereof include methyl methoxyacetate, ethyl methoxyacetate, butyl methoxyacetate, methyl ethoxyacetate, and ethyl ethoxyacetate. Examples of the 3-oxypropionic acid alkyl ester compound include methyl 3-oxipropionate and ethyl 3-oxipropionate, and specific examples thereof include methyl 3-methoxypropionate, ethyl 3-methoxypropionate, methyl 3-ethoxypropionate, and ethyl 3-ethoxypropionate. Examples of the 2-oxypropionic acid alkyl ester compound include methyl 2-oxypropionate, ethyl 2-oxypropionate, and propyl 2-oxypropionate, and specific examples thereof include methyl 2-methoxypropionate, ethyl 2-methoxypropionate, propyl 2-methoxypropionate, methyl 2-ethoxypropionate, and ethyl 2-ethoxypropionate.

Examples of the ether compound include diethylene glycol dimethyl ether, tetrahydrofuran, ethylene glycol monomethyl ether, ethylene glycol monoethyl ether, methyl cellosolve acetate, ethyl cellosolve acetate, diethylene glycol monomethyl ether, diethylene glycol monoethyl ether, diethylene glycol monobutyl ether, propylene glycol monomethyl ether, propylene glycol monomethyl ether acetate, diethylene glycol monoethyl ether acetate (also referred to as “ethyl carbitol acetate”), diethylene glycol monobutyl ether acetate (also referred to as “butyl carbitol acetate”), propylene glycol monoethyl ether acetate, and propylene glycol monopropyl ether acetate.

Examples of the ketone compound include methyl ethyl ketone, cyclohexanone, 2-heptanone, and 3-heptanone.

Suitable examples of the aromatic hydrocarbon compound include toluene and xylene.

In a case of containing a solvent, the polymerizable composition according to the present disclosure may contain only one kind of the solvent, but from the viewpoint of further improving the solubility of the components contained in the polymerizable composition and the coating surface state in a case of coating, the polymerizable composition according to the present disclosure may contain two or more kinds of the solvents.

In a case where the polymerizable composition according to the present disclosure contains two or more kinds of the solvents, it is preferable that the polymerizable composition according to the present disclosure contains two or more kinds of solvents selected from the group consisting of methyl 3-ethoxypropionate, ethyl 3-ethoxypropionate, ethyl cellosolve acetate, ethyl lactate, diethylene glycol dimethyl ether, butyl acetate, methyl 3-methoxypropionate, 2-heptanone, cyclohexanone, ethyl carbitol acetate, butyl carbitol acetate, propylene glycol methyl ether, and propylene glycol methyl ether acetate.

In a case where the polymerizable composition according to the present disclosure contains a solvent, a content of the solvent is not particularly limited, and can be appropriately set according to the purpose.

In a case where the polymerizable composition according to the present disclosure contains an organic solvent as the solvent, a content of the organic solvent is, for example, preferably 10% by mass to 80% by mass and more preferably 15% by mass to 60% by mass with respect to the total mass of the polymerizable composition.

### [Surfactant]

The polymerizable composition according to the present disclosure may contain a surfactant.

In a case where the polymerizable composition according to the present disclosure contains a surfactant, for example, the coating surface state in a case of coating and adhesiveness to a base material in a case of forming a film may be further improved.

Examples of the surfactant include surfactants described in paragraph of JP4502784B and paragraphs to of JP2009-237362A.

Examples of the surfactant include a fluorine-based surfactant, a silicone-based surfactant, a nonionic surfactant, an anionic surfactant, a cationic surfactant, and an amphoteric surfactant.

Among these, as the surfactant, at least one selected from the group consisting of a fluorine-based surfactant, a silicone-based surfactant, a nonionic surfactant, and an anionic surfactant is preferable.

As the fluorine-based surfactant, for example, it is preferable to use an acrylic compound, which has a molecular structure having a functional group including a fluorine atom and in which, by applying heat to the molecular structure, the functional group including a fluorine atom is broken to volatilize a fluorine atom. In addition, as the fluorine-based surfactant, for example, it is also preferable to use a copolymer of a fluorine atom-containing vinyl ether compound having a fluorinated alkyl group or a fluorinated alkylene ether group, and a hydrophilic vinyl ether compound. In addition, as the fluorine-based surfactant, for example, a block polymer can also be used. In addition, as the fluorine-based surfactant, for example, it is also preferable to use a fluorine-containing polymer compound including a repeating unit derived from a (meth)acrylate compound having a fluorine atom and a repeating unit derived from a (meth)acrylate compound having 2 or more (preferably 5 or more) alkyleneoxy groups [preferably an ethyleneoxy group and/or a propyleneoxy group]. In addition, as the fluorine-based surfactant, for example, a fluorine-containing polymer having an ethylenically unsaturated bond-containing group in the side chain can also be used.

A commercially available product can be used as the fluorine-based surfactant.

Examples of the commercially available product of the fluorine-based surfactant include: MEGAFACE (registered trademark) F-171, F-172, F-173, F-176, F-177, F-141, F-142, F-143, F-144, F-437, F-475, F-477, F-479, F-482, F-551-A, F-552, F-554, F-555-A, F-556, F-557, F-558, F-559, F-560, F-561, F-565, F-563, F-568, F-575, F-780, EXP.MFS-330, R-41, R-41-LM, R-01, R-40, R-40-LM, RS-43, TF-1956, RS-90, R-94, RS-718K, RS-72-K, RS-101, RS-102, and DS-21 [all of which are manufactured by DIC Corporation]; FLUORAD FC430, FC431, and FC171 [all of which are manufactured by Sumitomo 3M Ltd.]; SURFLON (registered trademark)S-382, SC-101, SC-103, SC-104, SC-105, SC-1068, SC-381, SC-383, S-393, and KH-40 [all of which are manufactured by Asahi Glass Co., Ltd.]; POLYFOX PF636, PF656, PF6320, PF6520, and PF7002 [all of which are manufactured by OMNOVA Solutions Inc.]; and FTERGENT (registered trademark) 710FM, 610FM, 601AD, 601ADH2, 602A, 215M, 245F, 251, 212M, 250, 209F, 222F, 208G, 710LA, 710FS, 730 LM, 650AC, and 681 [all of which are manufactured by NEOS COMPANY LIMITED].

Examples of the silicone-based surfactant include a linear polymer including a siloxane bond and a modified siloxane polymer with an organic group introduced in the side chain and/or the terminal.

A commercially available product can be used as the silicone-based surfactant.

Examples of the commercially available product of the silicone-based surfactant include DC3PA, SH7PA, DC11PA, SH21PA, SH28PA, SH29PA, SH30PA, SH8400, and DOWSIL (registered trademark) 8032 ADDITIVE [all manufactured by Dow Corning Corporation]; X-22-4952, X-22-4272, X-22-6266, KF-351A, K354L, KF-355A, KF-945, KF-640, KF-642, KF-643, X-22-6191, X-22-4515, KF-6004, KP-341, KF-6001, and KF-6002 [all manufactured by Shin-Etsu Chemical Co., Ltd.]; F-4440, TSF-4300, TSF-4445, TSF-4460, and TSF-4452 [all manufactured by Momentive Performance Materials Japan LLC]; and BYK307, BYK323, and BYK330 [all manufactured by BYK-Chemie GmbH].

Examples of the nonionic surfactant include glycerol, trimethylolpropane, trimethylolethane, and ethoxylates thereof (for example, glycerol ethoxylate) and propoxylates thereof (for example, glycerol propoxylate).

In addition, examples of the nonionic surfactant include polyoxyethylene alkyl ether, polyoxyethylene alkyl aryl ether, a polyoxyethylene-oxypropylene block copolymer, sorbitan fatty acid ester, polyoxyethylene sorbitan fatty acid ester, polyoxyethylene sorbitol fatty acid ester, glycerin fatty acid ester, polyoxyethylene fatty acid ester, and polyoxyethylene alkylamine.

A commercially available product can be used as the nonionic surfactant.

Examples of the commercially available product of the nonionic surfactant include PLURONIC (registered trademark) L10, L31, L61, L62, 10R5, 17R², and 25R² [all manufactured by BASF SE]; TETRONIC (registered trademark) 304, 701, 704, 901, 904, and 150R¹ [all manufactured by BASF SE]; Solsperse (registered trademark) 20000 [manufactured by Nippon Lubrizol Corporation]; NCW-101, NCW-1001, and NCW-1002 [all manufactured by FUJIFILM Wako Pure Chemical Corporation]; Pionin D-6112, D-6112-W, and D-6315 [all manufactured by TAKEMOTO OIL & FAT Co., Ltd.]; OLFINE (registered trademark) E1010 [all manufactured by Nissin Chemical Co., Ltd.], and SURFYNOL (registered trademark) 104, 400, and 440 [all manufactured by Nissin Chemical Co., Ltd.].

Examples of the anionic surfactant include a fatty acid salt, an alkyl sulfuric acid ester salt, an alkylbenzene sulfonate, an alkylnaphthalene sulfonate, a dialkyl sulfosuccinate, an alkyl diaryl ether disulfonate, an alkyl phosphate, a polyoxyethylene alkyl ether sulfate, a polyoxyethylene alkyl aryl ether sulfate, a naphthalenesulfonic formalin condensate, a polyoxyethylene alkyl phosphate ester salt, a glycerol borate fatty acid ester, and a polyoxyethylene glycerol fatty acid ester.

In a case where the polymerizable composition according to the present disclosure contains a surfactant, the polymerizable composition according to the present disclosure may contain only one kind of the surfactant, or may contain two or more kinds thereof.

In a case where the polymerizable composition according to the present disclosure contains a surfactant, a content of the surfactant is not particularly limited, but for example, preferably 0.01% by mass to 3% by mass, more preferably 0.05% by mass to 1% by mass, and still more preferably 0.1% by mass to 0.8% by mass with respect to the total solid content mass of the polymerizable composition.

### [Other Components]

The polymerizable composition according to the present disclosure may contain a component other than the above-described components (so-called other components) as necessary, within a range in which the effect of the present disclosure is not impaired.

Examples of the other components include various additives.

Examples of the additive include additives such as a colorant, a preservative, an antibacterial agent, and an antistatic agent.

### [Resin Composition]

The resin composition according to the present disclosure contains the compound according to the present disclosure and a resin.

### [Compound According to Present Disclosure]

The resin composition according to the present disclosure contains the compound according to the present disclosure.

The details of the compound according to the present disclosure are as described above, and thus the description thereof will be omitted.

The resin composition according to the present disclosure may contain only one kind of the compound according to the present disclosure, or may contain two or more kinds thereof.

A content of the compound according to the present disclosure in the resin composition according to the present disclosure is not particularly limited, and can be appropriately set according to the purpose.

The content of the compound according to the present disclosure in the resin composition according to the present disclosure is, for example, preferably 0.01% by mass to 90% by mass with respect to the total solid content mass of the resin composition. The lower limit thereof is more preferably 0.05% by mass or more and still more preferably 0.1% by mass or more. The upper limit thereof is more preferably 85% by mass or less and still more preferably 50% by mass or less.

### [Resin]

The resin composition according to the present disclosure contains a resin.

The resin in the resin composition according to the present disclosure has the same meaning as the resin as the polymer compound in the polymerizable composition according to the present disclosure, and preferred aspects thereof are also the same, so that the description thereof will be omitted here.

The resin composition according to the present disclosure may contain only one kind of the resin, or may contain two or more kinds thereof.

A content of the resin in the resin composition according to the present disclosure is not particularly limited, but for example, preferably 1% by mass to 99.9% by mass with respect to the total solid content mass of the resin composition. The lower limit thereof is more preferably 5% by mass or more and still more preferably 7% by mass or more. The upper limit thereof is more preferably 99% by mass or less and still more preferably 95% by mass or less.

### [Other Components]

The resin composition according to the present disclosure may contain a component other than the compound according to the present disclosure and the resin (so-called other components) as necessary. Examples of the other components include the components described in the polymerizable composition according to the present disclosure above. Specific examples thereof include a polymerizable compound, a polymerization initiator, an ultraviolet absorber, a solvent, and a surfactant. The details of these components are as described above, so that the description thereof will be omitted here.

In addition, the resin composition according to the present disclosure may contain various additives as necessary, within a range in which the effect of the present disclosure is not impaired. Examples of the additive include additives such as a colorant, a preservative, an antibacterial agent, and an antistatic agent.

### [Polymer]

The polymer according to the present disclosure contains a constitutional unit derived from the compound according to the present disclosure.

The polymer according to the present disclosure may be a homopolymer of the compound according to the present disclosure, or a copolymer of the compound according to the present disclosure and other compounds.

For example, the polymer according to the present disclosure may be a copolymer of the compound according to the present disclosure and the polymerizable compound, which is obtained from the above-described polymerizable composition according to the present disclosure, containing the compound according to the present disclosure ad the polymerizable compound; or may be a copolymer of the compound according to the present disclosure, the polymerizable compound, and the ultraviolet absorber, which is obtained from the above-described polymerizable composition according to the present disclosure, containing the compound according to the present disclosure, the polymerizable compound, and the ultraviolet absorber.

The details of the compound according to the present disclosure, the polymerizable compound, and the ultraviolet absorber are as described above, and thus the description thereof will be omitted here.

A weight-average molecular weight of the polymer according to the present disclosure is not particularly limited, but for example, preferably 5,000 to 80,000, more preferably 10,000 to 60,000, and still more preferably 10,000 to 40,000.

The polymer according to the present disclosure may contain only one kind of the constitutional unit derived from the compound according to the present disclosure, or may contain two or more kinds thereof.

A content of the constitutional unit derived from the compound according to the present disclosure in the polymer according to the present disclosure is not particularly limited, but for example, preferably 0.01% by mass to 100% by mass with respect to all constitutional units of the polymer. The lower limit thereof is more preferably 0.02% by mass or more and still more preferably 0.1% by mass or more. The upper limit thereof is more preferably 50% by mass or less and still more preferably 10% by mass or less.

In a case where the polymer according to the present disclosure contains a constitutional unit derived from the polymerizable compound, a content of the constitutional unit derived from the polymerizable compound is not particularly limited, but for example, preferably 20% by mass or more and less than 100% by mass with respect to all constitutional units of the polymer. The lower limit thereof is more preferably 30% by mass or more and still more preferably 50% by mass or more. The upper limit thereof is more preferably 99.99% by mass or less and still more preferably 99.9% by mass or less.

In a case where the polymer according to the present disclosure contains a constitutional unit derived from the ultraviolet absorber, a content of the constitutional unit derived from the ultraviolet absorber is not particularly limited, but for example, preferably 0.01% by mass to 90% by mass with respect to all constitutional units of the polymer. The lower limit thereof is more preferably 0.02% by mass or more and still more preferably 0.1% by mass or more. The upper limit thereof is more preferably 50% by mass or less and still more preferably 10% by mass or less.

### [Cured Substance]

The cured substance according to the present disclosure is a cured substance of the polymerizable composition according to the present disclosure.

The cured substance according to the present disclosure has a maximal absorption wavelength preferably in a range of 550 nm to 610 nm, more preferably in a range of 555 nm to 605 nm, and still more preferably in a range of 560 nm to 605 nm.

In a visible absorption spectrum of the cured substance according to the present disclosure, a width of an absorption spectrum at an absorbance corresponding to ½ of the maximum absorbance (so-called a half-width) is preferably 60 nm or less, and more preferably 40 nm or less.

In addition, in the visible absorption spectrum of the cured substance according to the present disclosure, a width of an absorption spectrum at an absorbance corresponding to ⅛ of the maximum absorbance is preferably 100 nm or less, and more preferably 80 nm or less.

The maximal absorption wavelength and width of the absorption spectrum of the cured substance according to the present disclosure are measured using a spectrophotometer. As a specific measuring method thereof, methods described in Examples below can be referred to.

The cured substance according to the present disclosure is obtained by curing the polymerizable composition according to the present disclosure.

A forming method of the cured substance according to the present disclosure is not particularly limited, and a known method can be adopted.

For example, the cured substance according to the present disclosure is obtained by forming a polymerizable composition layer using the polymerizable composition according to the present disclosure, and then curing the formed polymerizable composition layer by applying energy to the polymerizable composition layer. The polymerizable composition layer may be formed on a desired support.

A method of applying energy to the polymerizable composition layer is not particularly limited, and examples thereof include light irradiation and heating. Among these, as the method of applying energy to the polymerizable composition layer, light irradiation is preferable, and ultraviolet irradiation is more preferable.

In a case where the energy application is light irradiation, it is preferable that the polymerizable composition according to the present disclosure contains the photopolymerization initiator.

In a case where the polymerizable composition layer is cured by ultraviolet irradiation, for example, an ultraviolet lamp can be used for the ultraviolet irradiation.

A light irradiation amount of ultraviolet rays is not particularly limited, but for example, preferably 10 mJ/cm² to 1,000 mJ/cm². In a case where the light irradiation amount of ultraviolet rays is within the above-described range, the curing of the polymerizable composition layer tends to be more suitably is carried out.

In a case of irradiation with ultraviolet rays, for the purpose of suppressing curing inhibition caused by oxygen and further accelerating surface curing of the polymerizable composition layer, an inert gas such as nitrogen gas may be sent into a spatial domain where the irradiation with ultraviolet rays is carried out to replace the air in the ultraviolet irradiated region with the inert gas to reduce an oxygen concentration.

The oxygen concentration in the spatial domain where the irradiation with ultraviolet rays is carried out is not particularly limited, but for example, preferably 1% or less.

For the purpose of promoting the curing reaction of the polymerizable composition layer, a temperature at the time of curing the polymerizable composition layer may be increased. For example, the temperature at the time of curing the polymerizable composition layer is preferably 25° C. to 100° C., more preferably 30° C. to 80° C., and still more preferably 40° C. to 70° C.

In a case where the polymerizable composition according to the present disclosure contains a solvent, from the viewpoint of improving curing properties of the polymerizable composition layer, it is preferable that the polymerizable composition layer is dried in advance to reduce the amount of the solvent before the energy is applied to the polymerizable composition layer.

A drying method of the polymerizable composition layer is not particularly limited, and a known drying method can be adopted.

Examples of the drying method of the polymerizable composition layer include a method of blowing hot air onto the composition layer, a method of passing the composition layer through a drying zone controlled at a predetermined temperature, and a method of transporting the composition layer by a transport roll equipped with a heater.

In addition, the cured substance according to the present disclosure is also obtained, for example, by filling a desired molding mold with the polymerizable composition, and then curing the filled polymerizable composition by applying energy to the polymerizable composition. A method of applying the energy in this case is preferably heating.

In a case where the energy application is heating, it is preferable that the polymerizable composition according to the present disclosure contains the thermal polymerization initiator.

In a case where the energy application is heating, a heating time is not particularly limited, but for example, preferably 30 seconds to 1,000 seconds, more preferably 30 seconds to 500 seconds, and still more preferably 60 seconds to 300 seconds.

The atmosphere in a case of thermally polymerizing the polymerizable composition by the heating may be an air atmosphere or an inert gas atmosphere such as a nitrogen gas; and for example, from the viewpoint of curing properties of the polymerizable composition, an inert gas atmosphere is preferable, and an inert gas atmosphere in which the oxygen concentration is 1% or less is more preferable.

In addition, the cured substance according to the present disclosure is also obtained, for example, by the following method. After forming the polymerizable composition layer using the polymerizable composition according to the present disclosure, energy is applied to the formed polymerizable composition layer under conditions in which the polymerizable composition layer is to be a semi-cured substance before being completely cured, thereby obtaining a semi-cured substance of the polymerizable composition layer. Next, the obtained semi-cured substance is put into a desired molding mold, and energy is applied under conditions for complete curing, thereby obtaining the cured substance according to the present disclosure. Such a method is preferable from the viewpoint that a cured substance having an optional shape can be easily obtained.

Specific examples of applications of the cured substance according to the present disclosure include an optical filter (for example, a color correction filter for a display) and a lens for glasses.

The cured substance according to the present disclosure is preferably an optical filter.

In a case where the cured substance according to the present disclosure is an optical filter, it is preferable that the cured substance according to the present disclosure does not have an absorption maximal in a wavelength range other than a wavelength range of 550 nm to 610 nm. The optical filter having such characteristics is suitable as an optical filter for a liquid crystal display device, which has a wide color reproduction range and a high brightness.

In a case where the cured substance according to the present disclosure is an optical filter, a thickness of the cured substance according to the present disclosure is not particularly limited, but for example, preferably 5 μm to 2,500 μm and more preferably 20 μm to 1,000 μm.

In a case where the thickness of the cured substance according to the present disclosure is within the above-described range, a desired visible light transmittance is easily obtained, and the cured substance is also easily handled as the optical filter.

In a case where the cured substance according to the present disclosure is an optical filter, a content of the compound according to the present disclosure in the cured substance according to the present disclosure is not particularly limited, but for example, preferably 0.005 mmol/m² to 0.1 mmol/m² and more preferably 0.01 mmol/m² to 0.05 mmol/m².

The content of the compound according to the present disclosure in the cured substance according to the present disclosure can be controlled by preparing the polymerizable composition according to the present disclosure.

A shape of the cured substance according to the present disclosure can be controlled by using a molding mold in a case of curing the polymerizable composition according to the present disclosure. Since the shape of the cured substance according to the present disclosure can be easily controlled according to the application, the use place, and the like, the cured substance can be applied to products in various fields. For example, the cured substance according to the present disclosure can also be applied to a dome-shaped optical filter, a lens for glasses, goggles, and the like.

### [Laminate]

The laminate according to the present disclosure includes a support and the cured substance according to the present disclosure.

Since the laminate according to the present disclosure includes the cured substance according to the present disclosure, that is, the cured substance which is formed of the polymerizable composition according to the present disclosure, containing the compound according to the present disclosure, the bleed-out of the compound according to the present disclosure is less likely to occur.

The support may be transparent or may not be transparent, but is preferably transparent.

In the present disclosure, “transparent” means that an average transmittance of visible light having a wavelength of 450 nm to 750 nm is 80% or more, preferably 90% or more.

In the present disclosure, the average transmittance of visible light is obtained by performing spectral spectrum measurement using a spectrophotometer, and calculating a value from light quantity of a transmitted light with respect to light quantity of an incidence light of the visible light in the above-described wavelength range. As the spectrophotometer, for example, an ultraviolet-visible spectrophotometer (model number: UV-1800) manufactured by Shimadzu Corporation can be suitably used. However, the spectrophotometer is not limited thereto.

The support is not particularly limited, and examples thereof include glass and a resin film.

Examples of a resin used as a material of the resin film include ester resins [for example, polyethylene terephthalate (PET), polyethylene naphthalate (PEN), polybutylene terephthalate (PBT), and polycyclohexanedimethylene terephthalate (PCT)], olefin resins [for example, polypropylene (PP) and polyethylene (PE)], polyvinyl chloride (PVA), cellulose triacetate (TAC), polymethyl methacrylate (PMMA), polystyrene (PS), and polycarbonate (PC).

Among these, as the resin used as a material of the resin film, for example, from the viewpoint of general-purpose properties, PET is preferable.

A thickness of the support is not particularly limited, and can be appropriately selected according to the use or purpose.

The thickness of the support can be, for example, in a range of 100 μm to 10 mm.

A shape of the support is not particularly limited, and can be appropriately selected according to the use or purpose.

For example, the laminate according to the present disclosure may be a laminate including, on a lens-shaped support, an optical filter which is a lens-shaped cured substance according to the present disclosure.

The support may have peelability.

Examples of the support having peelability include a support in which one side or both sides have been subjected to a surface treatment using a peeling treatment agent (so-called easy peeling treatment).

Examples of the peeling treatment agent include a silicone-based peeling treatment agent (for example, silicone), a wax-based peeling treatment agent (for example, paraffin wax), and a fluorine-based peeling treatment agent (for example, a fluorine-based resin).

The maximal absorption wavelength of the laminate according to the present disclosure is preferably in a range of 550 nm to 610 nm, and more preferably in a range of 580 nm to 605 nm.

In a visible absorption spectrum of the laminate according to the present disclosure, a width of an absorption spectrum at an absorbance corresponding to ½ of the maximum absorbance (so-called a half-width) is preferably 60 nm or less, and more preferably 40 nm or less.

In addition, in the visible absorption spectrum of the laminate according to the present disclosure, a width of an absorption spectrum at an absorbance corresponding to ⅛ of the maximum absorbance is preferably 100 nm or less, and more preferably 80 nm or less.

The maximal absorption wavelength and width of the absorption spectrum of the laminate according to the present disclosure are measured using a spectrophotometer. As a specific measuring method thereof, methods described in Examples below can be referred to.

The laminate according to the present disclosure may have a two-layer structure of the support and the cured substance according to the present disclosure, or may have a three or more-layer structure including the support, the cured substance according to the present disclosure, and other layers.

Examples of the other layers include an adhesive layer, a surface protective layer (for example, an overcoat layer and a hard coat layer), a reflective layer (for example, a dielectric multi-layer film and a photonic crystal film), and a colored layer.

A manufacturing method of the laminate according to the present disclosure is not particularly limited, and a known manufacturing method can be adopted.

The laminate according to the present disclosure can be manufactured, for example, by applying the polymerizable composition according to the present disclosure onto the support to form a polymerizable composition layer, and then curing the formed polymerizable composition layer by applying energy to the polymerizable composition layer.

In addition, the laminate according to the present disclosure can be manufactured, for example, by the following method. The cured substance according to the present disclosure is obtained by filling a desired molding mold with the polymerizable composition, and then curing the filled polymerizable composition by applying energy to the polymerizable composition. Next, the obtained cured substance according to the present disclosure is attached to the support to manufacture the laminate according to the present disclosure.

### EXAMPLES

Hereinafter, the present disclosure will be described in detail according to Examples. However, the present disclosure is not limited to Examples below.

### Example 1: Synthesis of Exemplary Compound 1-13

An exemplary compound 1-13 was synthesized as follows.

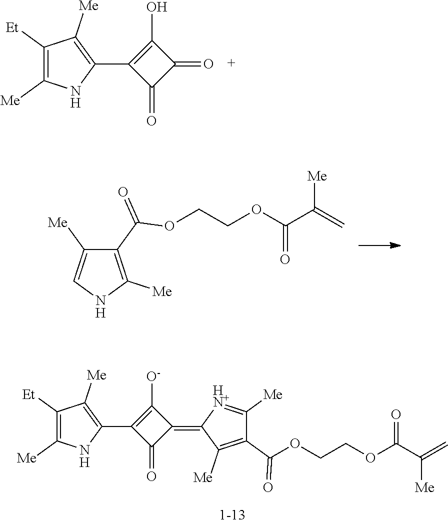

0.53 g of 3-(2-methacryloyloxyethoxycarbonyl)-2,4-dimethylpyrrole, 0.55 g of 3-(3-ethyl-2,4-dimethylpyrrol-2-yl)-4-hydroxy-3-cyclobuten-1,2-dione, 4 mL of 2-butanol, and 8 mL of toluene were added to a 50 mL eggplant flask equipped with a Dean-Stark trap. Next, the Dean-Stark trap was filled with a solution of 2-butanol/toluene=½ (volume ratio). The mixture in the eggplant flask was heated and refluxed for 3 hours, and generated water to be generated was distilled off in the Dean-Stark trap. The reaction mixture was cooled to room temperature, and concentrated under reduced pressure. The residue was purified by a silica gel chromatograph. 3 mL of methanol was added to the obtained compound, and the mixture was stirred at room temperature (25° C.) for 30 minutes. The precipitate was collected by filtration, and dried to obtain 0.47 g of the exemplary compound 1-13.

In a case where a structure of the exemplary compound 1-13 was obtained by 1H-NMR, it was confirmed to be the above-described structure.

The NMR data of the exemplary compound 1-13 is shown below.

(Exemplary compound 1-13)

¹H-NMR (DMSO-d₆) δ=1.03 (t, 3H), 1.89 (s, 3H), 2.41 (q, 2H), 2.43 (s, 3H), 2.53 (s, 3H), 2.58 (s, 3H), 2.72 (s, 3H), 4.42 (m, 4H), 5.71 (s, 1H), 6.05 (s, 1H), 11.55 (s, 1H), 11.91 (s, 1H)

### Example 2: Synthesis of Exemplary Compound 1-30

An exemplary compound 1-30 was synthesized as follows.

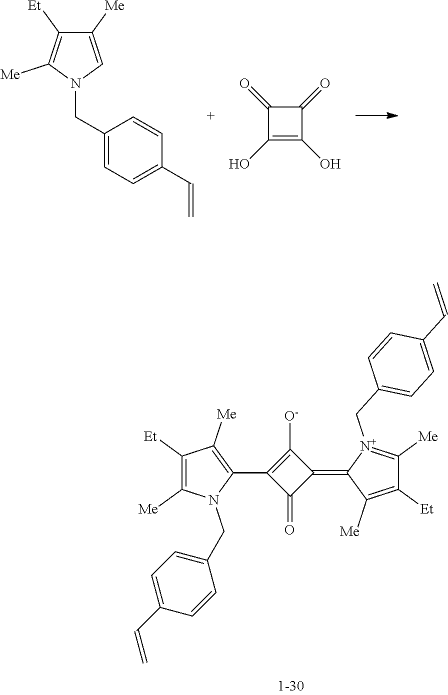 [SMILES: C=Cc1ccc(Cn2cc(C)c(CC)c2C)cc1 ; O=c1c(O)c(O)c1=O ; C=Cc1ccc(Cn2c(C)c(CC)c(C)c2C2=C([O-])/C(=C3/C(C)=C(CC)C(C)=[N+]3Cc3ccc(C=C)cc3)C2=O)cc1]

1.0 g of 3-ethyl-2,4-dimethyl-1-(4-vinylbenzyl) pyrrole, 250 mg of 3,4-dihydroxy-3-cyclobutene-1,2-dione, 3 mL of n-butanol, and 6 mL of toluene were added to a 50 mL eggplant flask equipped with a Dean-Stark trap. Next, the Dean-Stark trap was filled with a solution of n-butanol/toluene=½ (volume ratio). The mixture in the eggplant flask was heated and refluxed for 2 hours, and generated water to be generated was distilled off in the Dean-Stark trap. The reaction mixture was cooled to room temperature (25° C.), and concentrated under reduced pressure. The residue was purified by a silica gel chromatograph. 10 mL of methanol was added to the obtained compound, and the mixture was heated and refluxed for 10 minutes. The mixture was cooled to room temperature (25° C.), and the precipitate was collected by filtration and dried to obtain 0.5 g of the exemplary compound 1-30.

In a case where a structure of the exemplary compound 1-30 was obtained by 1H-NMR, it was confirmed to be the above-described structure.

The NMR data of the exemplary compound 1-30 is shown below.

### (Exemplary Compound 1-30)

¹H-NMR (CDCl₃) δ=1.04 (t, 6H), 2.18 (s, 6H), 2.42 (q, 4H), 2.61 (s, 6H), 5.18 (d, 2H), 5.65 (d, 2H), 5.96 (s, 4H), 6.61 (dd, 2H), 6.76 (d, 4H), 7.17 (d, 4H) (Example 3: synthesis of exemplary compound 1-53)

First, an intermediate 1-53a was synthesized as follows.

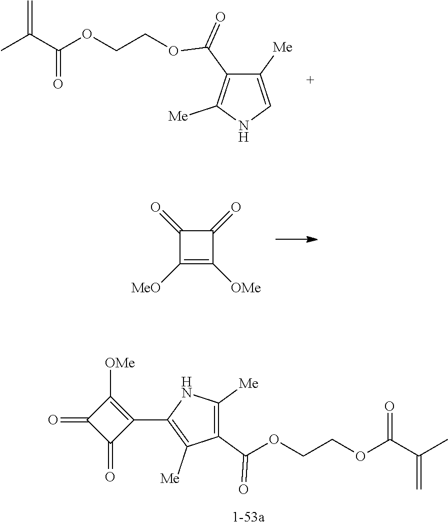 [SMILES: C=C(C)C(=O)OCCOC(=O)c1c(C)c[nH]c1C ; COc1c(OC)c(=O)c1=O ; C=C(C)C(=O)OCCOC(=O)c1c(C)[nH]c(-c2c(OC)c(=O)c2=O)c1C]

0.83 g of 3-(2-methacryloyloxyethoxycarbonyl)-2,4-dimethylpyrrole, 0.42 g of 3,4-dimethoxy-3-cyclobutene-1,2-dione, and 4 mL of acetic acid were added to a 50 mL eggplant flask. The mixture in the eggplant flask was stirred at 40° C. for 6 hours. The reaction mixture was cooled to room temperature (25° C.). The cooled reaction mixture was added to ethyl acetate and water to be liquid-separated. The organic layer was washed with a sodium bicarbonate aqueous solution, and concentrated under reduced pressure. The residue was purified by a silica gel chromatograph to obtain 0.27 g of the intermediate 1-53a. Next, an intermediate 1-53b was synthesized as follows.

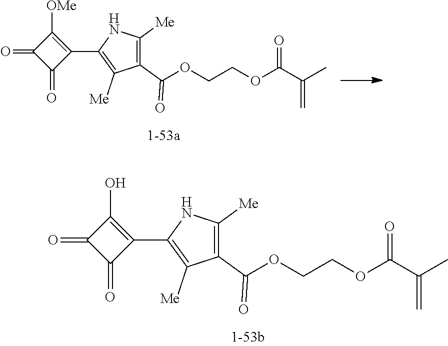 [SMILES: C=C(C)C(=O)OCCOC(=O)c1c(C)[nH]c(-c2c(OC)c(=O)c2=O)c1C ; C=C(C)C(=O)OCCOC(=O)c1c(C)[nH]c(-c2c(O)c(=O)c2=O)c1C]

0.25 g of 1-53a was added to a 50 mL eggplant flask with 2 mL of acetic acid, 1 mL of water, and 100 μL of concentrated hydrochloric acid. The mixture in the eggplant flask was stirred at 60° C. for 3 hours. The reaction mixture was cooled to room temperature (25° C.). The cooled reaction mixture was added to ethyl acetate and water to be liquid-separated. The organic layer was washed with a sodium bicarbonate aqueous solution, and concentrated under reduced pressure. The residue was purified by a silica gel chromatograph to obtain 0.10 g of the intermediate 1-53b.

Next, an exemplary compound 1-53 was synthesized as follows.

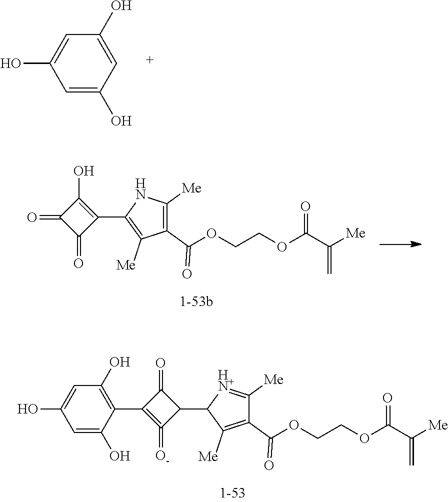

0.10 g of 1-53b, 0.04 g of phloroglucinol, 4 mL of 2-butanol, and 8 mL of toluene were added to a 50 mL eggplant flask equipped with a Dean-Stark trap. Next, the Dean-Stark trap was filled with a solution of 2-butanol/toluene=½ (volume ratio). The mixture in the eggplant flask was heated and refluxed for 3 hours, and generated water to be generated was distilled off in the Dean-Stark trap. The reaction mixture was cooled to room temperature (25° C.), and concentrated under reduced pressure. The residue was purified by a silica gel chromatograph. 3 mL of methanol was added to the obtained compound, and the mixture was stirred at room temperature (25° C.) for 30 minutes. The precipitate was collected by filtration, and dried to obtain 0.02 g of the exemplary compound 1-53.

MS (m/z)=456.1 ([M+1]⁺)

### Comparative Example 1: Comparative Compound R-1

An exemplary compound No. 3 described in JP2019-12159A was synthesized and used as a comparative compound R-1. The structure and NMR data of the comparative compound R-1 are shown below.

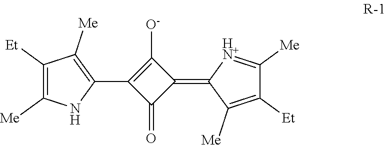

¹H-NMR (CDCl₃) δ=1.08 (t, 6H), 2.34 (s, 6H), 2.43 (q, 4H), 2.56 (s, 6H), 9.94 (br.s, 2H)

### Comparative Example 2: comparative compound R-2

An exemplary compound XXIIa described in JP2013-535558A was synthesized and used as a comparative compound R-2. The structure and NMR data of the comparative compound R-2 are shown below.

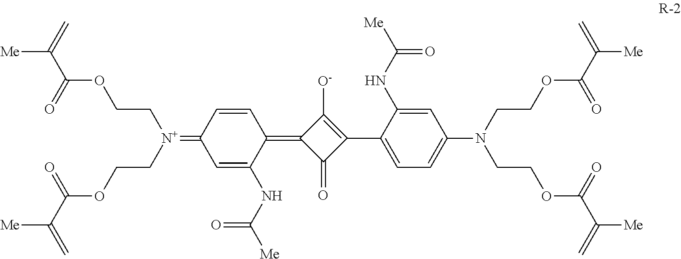 [SMILES: C=C(C)C(=O)OCCN(CCOC(=O)C(=C)C)c1ccc(C2=C([O-])/C(=C3\C=CC(=[N+](CCOC(=O)C(=C)C)CCOC(=O)C(=C)C)C=C3NC(C)=O)C2=O)c(NC(C)=O)c1]

¹H-NMR (CDCl₃) δ=1.92 (s, 12H), 2.36 (s, 6H), 3.89 (t, 8H), 4.43 (t, 8H), 5.59 (s, 4H), 6.09 (s, 4H), 6.62 (dd, 2H), 8.41 (d, 2H), 8.49 (d, 2H), 12.11 (s, 2H)

### Comparative Example 3: Comparative Compound R-3

The compound described in Comparative Example 2 (that is, the comparative compound R-2), in which a polymerizable group was not provided, was synthesized and used as a comparative compound R-3. The structure and NMR data of the comparative compound R-3 are shown below.

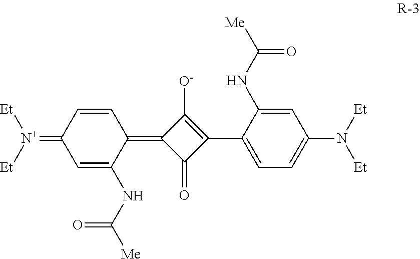 [SMILES: CCN(CC)c1ccc(C2=C([O-])/C(=C3\C=CC(=[N+](CC)CC)C=C3NC(C)=O)C2=O)c(NC(C)=O)c1]

¹H-NMR (CDCl₃) δ=12.11 (s, 2H), 8.43 (d, 2H), 8.26 (d, 2H), 6.46 (dd, 2H), 3.53 (q, 8H), 2.36 (s, 6H), 1.29 (t, 12H)

### <Spectral Characteristics>

An absorption spectrum of each of the compounds of Examples 1 to 3 and Comparative Examples 1 to 3 was measured according to the following procedure, and the maximal absorption wavelength (2max), the molar absorption coefficient (8), and the width of the absorption spectrum at an absorbance corresponding to ½ of the maximum absorbance (that is, the half-width) were obtained. The results are shown in Table 1.

Using chloroform as a solvent, a solution in which the compound was dissolved was prepared such that the concentration of the compound in the chloroform was 5×10⁻⁶ mol/L (liter; the same applies hereinafter). Each prepared solution was put into a 1 cm cell, and an absorption spectrum was measured using a spectrophotometer [product name: U-4100, manufactured by Hitachi, Ltd.] as a measurement device.

TABLE 1

| | | | ε | Half- |
|---|---|---|---|---|
| | Compound | λmax | [L/(mol · cm)] | width |
| Example 1 | Exemplary compound | 556 nm | 185,000 | 25 nm |
| | 1-13 | | | |
| Example 2 | Exemplary compound | 591 nm | 158,000 | 29 nm |
| | 1-30 | | | |
| Example 3 | Exemplary compound | 560 nm | 193,000 | 26 nm |
| | 1-53 | | | |
| Comparative | Comparative | 564 nm | 238,000 | 23 nm |
| Example 1 | compound R-1 | | | |
| Comparative | Comparative | 667 nm | 363,000 | 27 nm |
| Example 2 | compound R-2 | | | |
| Comparative | Comparative | 676 nm | 356,000 | 27 nm |
| Example 3 | compound R-3 | | | |

### <Solubility>

The solubility of each compound of Examples 1 to 3 and Comparative Examples 1 to 3 in an organic solvent was measured according to the following procedure.

20.0 mg of the compound was weighed and placed in a container, 2 mL of ethyl acetate was added thereto, and the mixture was stirred at 40° C. for 10 minutes. After the stirring, the presence or absence of undissolved residue of the compound was visually observed. The fact that no undissolved residue of the compound was observed means that the solubility of the compound was 1% by mass or more.

The exemplary compound 1-13, exemplary compound 1-30, and exemplary compound 1-53, which were the compound according to the present disclosure, were completely dissolved in ethyl acetate as the organic solvent, and no undissolved residue was observed. On the other hand, the comparative compound R-1, the comparative compound R-2, and the comparative compound R-3 were not completely dissolved in ethyl acetate as the organic solvent, and the undissolved residue was observed.

From the above results, it was found that the compound according to the present disclosure had excellent solubility in an organic solvent, as compared with the squarylium compound in the related art, which did not have a polymerizable group. In addition, it was also found that the compound according to the present disclosure had excellent solubility in an organic solvent, as compared with the squarylium compound in the related art, which had a polymerizable group.

### Example 4

### 1. Preparation of Polymerizable Composition P1

A polymerizable composition P1 having the following formulation was prepared.

The polymerizable composition P1 was obtained by mixing the following components.

### Formulation of Polymerizable Composition P1

| | | |
|---|---|---|
| Exemplary compound 1-13 | 0.3 | parts by mass |
| Resin | 10.6 | parts by mass |
| [benzyl methacrylate/acrylic acid copolymer (monomer mass ratio: 70/30)] | | |
| Polymerizable compound | 31.7 | parts by mass |
| [dipentaerythritol hexaacrylate, manufactured by Tokyo Chemical Industry Co., Ltd.] | | |
| Photopolymerization initiator (1) | 6.3 | parts by mass |
| [IRGACURE (registered trademark) OXE01, manufactured by BASF SE] | | |
| Photopolymerization initiator (2) | 2.1 | parts by mass |
| [2,4-diethylthioxanthone, manufactured by Tokyo Chemical Industry Co., Ltd.] | | |
| Solvent (1) | 28.6 | parts by mass |
| [propylene glycol monomethyl ether acetate, manufactured by FUJIFILM Wako Pure | | |
| Chemical Corporation] | | |
| Solvent (2) | 20.4 | parts by mass |
| [cyclohexanone, manufactured by FUJIFILM Wako Pure Chemical Corporation] | | |

### 2. Production of Cured Film E1

The polymerizable composition P1 was applied onto a glass substrate having a size of 20 mm×20 mm by spin coating, thereby forming a polymerizable composition layer C1 which was a coating film of the polymerizable composition P1. The formed polymerizable composition layer C1 was dried at 80° C. for 3 minutes.

Next, the dried polymerizable composition layer C1 was exposed to UV-LED black light [product name: LLHA 165×25-22UV365YSIC, luminescence wavelength: 365 nm, manufactured by AITEC SYSTEM Co., Ltd.] to obtain a photocured film DI which was a semi-cured substance. The light irradiation was carried out with an exposure amount of 20 mJ/cm² on average in the exposure region, a gap of 100 μm from the coating film, and light quantity of 85 mJ/cm² as an integrated exposure amount in a case where the coating film was irradiated with light at 365 nm for 5 seconds.

Next, the photocured film DI was heated at 190° C. for 20 minutes to be completely cured, thereby obtaining a cured film E1.

### Comparative Example 4

### 1. Preparation of Polymerizable Composition P2

A polymerizable composition P2 was obtained in the same manner as in the preparation of the polymerizable composition P1, except that, in the preparation of the polymerizable composition P1, the “exemplary compound 1-13” was changed to the “comparative compound R-1”.

### 2. Production of Cured Film E2

A cured film E2 was obtained in the same manner as in the production of the cured film E1, except that, in the production of the cured film E1, the “polymerizable composition P1” was changed to the “polymerizable composition P2”.

### Comparative Example 5

### 1. Preparation of Polymerizable Composition P3

A polymerizable composition P3 was obtained in the same manner as in the preparation of the polymerizable composition P1, except that, in the preparation of the polymerizable composition P1, the “exemplary compound 1-13” was changed to the “comparative compound R-2”.

### 2. Production of Cured Film E3

A cured film E3 was obtained in the same manner as in the production of the cured film E1, except that, in the production of the cured film E1, the “polymerizable composition P1” was changed to the “polymerizable composition P3”.

### Comparative Example 6

### 1. Preparation of Polymerizable Composition P4

A polymerizable composition P4 was obtained in the same manner as in the preparation of the polymerizable composition P1, except that, in the preparation of the polymerizable composition P1, the “exemplary compound 1-13” was changed to the “comparative compound R-3”.

### 2. Production of Cured Film E4

A cured film E4 was obtained in the same manner as in the production of the cured film E1, except that, in the production of the cured film E1, the “polymerizable composition P1” was changed to the “polymerizable composition P4”. However, since the “comparative compound R-3” had extremely low solubility in the organic solvent [that is, the solvent (1) and the solvent (2)], the obtained photocured film D4 and cured film E4 were not films that could be evaluated for the spectral characteristics, so that the following evaluation tests were not performed.

### <Spectral Characteristics>

Absorption spectra of the photocured film DI to the photocured film D3 and the cured film E1 to the cured film E3 were measured using a spectrophotometer [product name: U-4100, manufactured by Hitachi, Ltd.] as a measuring device. The maximal absorption wavelength (λmax), the absorbance at the maximal absorption wavelength (λmax), and the half-width were obtained.

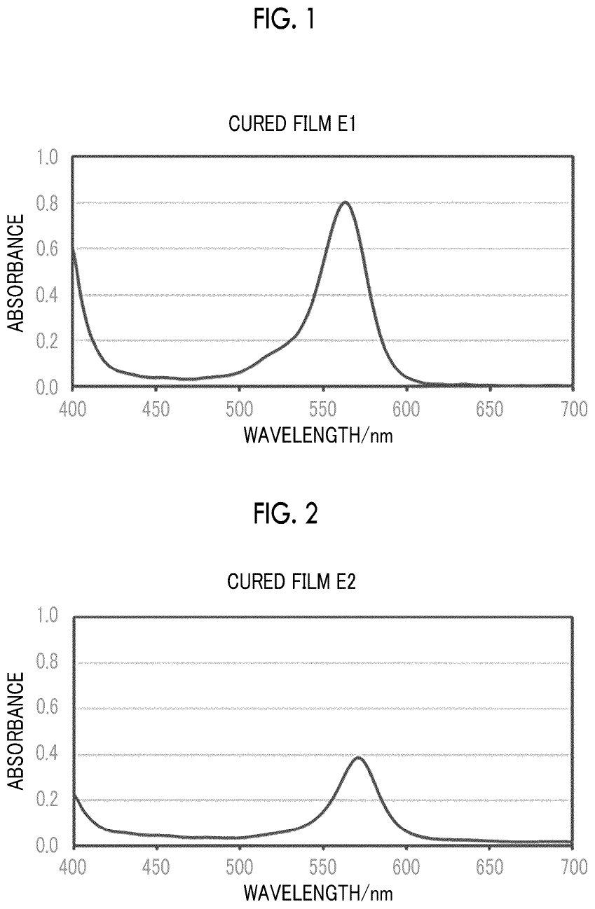

The absorption spectra of the cured film E1, the cured film E2, and the cured film E3 are shown in FIGS. 1, 2, and 3, respectively. In addition, Table 2 shows the maximal absorption wavelength (λmax), the absorbance at the maximal absorption wavelength (λmax), and the half-width of the photocured film DI to the photocured film D3 and the cured film E1 to the cured film E3.

In the photocured film D3, since the absorbance at the maximal absorption wavelength (λmax) was extremely low, it was difficult to obtain the half-width, so that the evaluation could not be performed. In Table 2, it is indicated as “-”.

TABLE 2

| | Cured film | λmax | Absorbance at λmax | Half-width |
|---|---|---|---|---|
| Example 4 | D1 | 563 nm | 0.88 | 33 nm |
| | E1 | 563 nm | 0.80 | 33 nm |
| Comparative | D2 | 570 nm | 0.88 | 28 nm |
| Example 4 | E2 | 571 nm | 0.39 | 32 nm |
| Comparative | D3 | 678 nm | 0.10 | — |
| Example 5 | E3 | 678 nm | 0.19 | 46 nm |

As shown in Table 2 and FIG. 1, in a case where the compound according to the present disclosure was used, no significant change in absorbance and half-width of the cured film was found before and after the curing by heating.

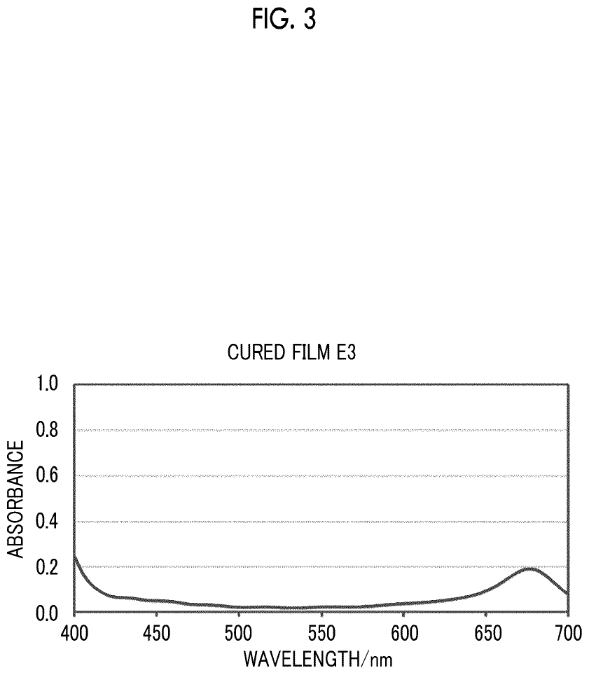

On the other hand, as shown in Table 2 and FIG. 2, in a case of using the squarylium compound in the related art, in which the pyrrole ring not having a polymerizable group was used as the basic nucleus, a significant decrease in absorbance of the cured film and widening of the half-width were found before and after the curing by heating. In addition, as shown in Table 2 and FIG. 3, in a case where the squarylium compound in the related art, having a polymerizable group, was used, it was found that the absorbance of the cured film was significantly reduced. Furthermore, in a case where the squarylium compound in the related art, not having a polymerizable group, was used, since the solubility in the organic solvent was extremely low, a film in which the spectral characteristics could be evaluated was not obtained.

It is considered that, since the compound according to the present disclosure has excellent solubility in an organic solvent because the compound has the specific structure having a polymerizable group, the bleed-out from the formed film is suppressed. In addition, it is considered that, since the compound according to the present disclosure has a polymerizable group and is fixed to the cured film by the polymerization reaction due to light irradiation, in comparison with the squarylium compound in the related art, in which the pyrrole ring not having a polymerizable group is used as the basic nucleus, the bleed-out from the film is suppressed in the process of being completely cured by heating. From the above, it is presumed that the decrease in spectral characteristics is unlikely to occur in a case where the compound according to the present disclosure is used.

The disclosure of Japanese Patent Application No. 2022-054280 filed on Mar. 29, 2022 and the disclosure of Japanese Patent Application No. 2022-122246 filed on Jul. 29, 2022 are incorporated in the present specification by reference.

All documents, patent applications, and technical standards described in the present specification are incorporated herein by reference to the same extent as in a case of being specifically and individually noted that individual documents, patent applications, and technical standards are incorporated by reference.

What is claimed is:

1. A compound represented by Formula (X), 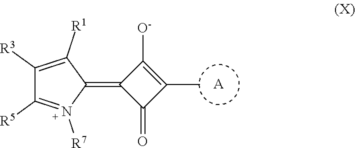
in Formula (X), R¹, R³, R⁵, and R⁷ each independently represent a hydrogen atom or a substituent, R¹ and R³, R³ and R⁵, and R⁵ and R⁷ each may be bonded to each other to form a ring, and a ring A represents an aromatic ring, provided that at least one substituent in Formula (X) represents a group which includes a polymerizable group having an ethylenically unsaturated bond.

2. The compound according to claim 1,
wherein the compound represented by Formula (X) is a compound represented by Formula (1), 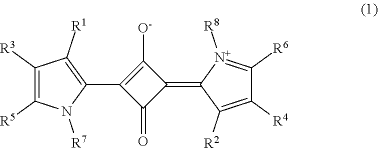
in Formula (1), R¹, R², R³, R⁴, R⁵, R⁶, R⁷, and R⁸ each independently represent a hydrogen atom or a substituent, and R¹ and R³, R² and R⁴, R³ and R⁵, R⁴ and R⁶, R⁵ and R⁷, and R⁶ and R⁸ each may be bonded to each other to form a ring, provided that at least one of R¹, R², R³, R⁴, R⁵, R⁶, R⁷, or R⁸ represents a group which includes a polymerizable group having an ethylenically unsaturated bond.

3. The compound according to claim 2,
wherein, in Formula (1), R¹, R², R³, R⁴, R⁵, and R⁶ are each independently a hydrogen atom, a halogen group, an alkyl group, an aryl group, an alkoxy group, an aryloxy group, an acyl group, an alkoxycarbonyl group, an aryloxycarbonyl group, a carbamoyl group, an acylamino group, an alkoxycarbonylamino group, a carbamoylamino group, or a cyano group, R⁷ and R⁸ are each independently a hydrogen atom, an alkyl group, or an aryl group, and at least one of R¹, R², R³, R⁴, R⁵, R⁶, R⁷, or R⁸ includes the polymerizable group having an ethylenically unsaturated bond.

4. The compound according to claim 2,
wherein, in Formula (1), at least one of R³ or R⁴ is a group represented by Formula (8), 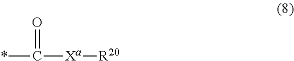
in Formula (8), X^{a} represents a single bond, an alkylene group, —O—, —S—, or —NR⁹—, where R⁹ represents a hydrogen atom or an alkyl group, R²⁰ represents a hydrogen atom or an alkyl group, and * represents a bonding position.

5. The compound according to claim 1,
wherein the group which includes a polymerizable group having an ethylenically unsaturated bond is a group including a group represented by Formula (2) or a group including a group represented by Formula (3), 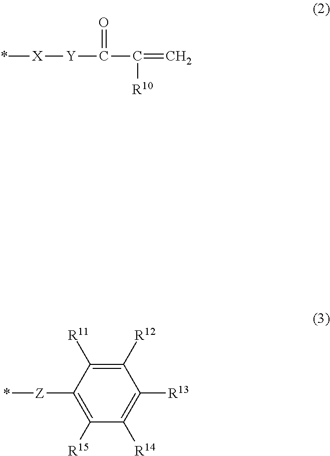
in Formula (2), X represents a single bond or an alkylene group, Y represents a single bond, —O—, or —NR¹⁶—, where R¹⁶ represents a hydrogen atom or an alkyl group, R¹⁰ represents a hydrogen atom or an alkyl group, and * represents a bonding position,
in Formula (3), R¹¹, R¹², R¹³, R¹⁴, and R¹⁵ each independently represent a hydrogen atom, a halogen group, an alkyl group, an alkoxy group, or a vinyl group, Z represents a single bond or an alkylene group, and * represents a bonding position, provided that at least one of R¹¹, R¹², R¹³, R¹⁴, R¹⁵ represents a vinyl group.

6. The compound according to claim 1,
wherein the compound is a squarylium coloring agent.

7. A polymerizable composition comprising:
the compound according to claim 1; and
a polymerizable compound.

8. The polymerizable composition according to claim 7,
wherein the polymerizable compound is at least one selected from the group consisting of a (meth)acrylic acid, a (meth)acrylic acid amide compound, a (meth)acrylic acid ester compound, and a styrene compound.

9. The polymerizable composition according to claim 7, further comprising:
an ultraviolet absorber.

10. The polymerizable composition according to claim 7, further comprising:
a polymer compound.

11. A resin composition comprising:
the compound according to claim 1; and
a resin.

12. A polymer comprising:
a constitutional unit derived from the compound according to claim 1.

13. A cured substance of the polymerizable composition according to claim 7.

14. The cured substance according to claim 13,
wherein the cured substance is an optical filter.

15. The cured substance according to claim 13,
wherein a maximal absorption wavelength is in a range of 550 nm to 610 nm.

16. A laminate comprising:
a support; and
the cured substance according to claim 13.

17. The laminate according to claim 16,
wherein a maximal absorption wavelength is in a range of 550 nm to 610 nm.

18. A production method of the compound according to claim 1, the production method comprising:
reacting a compound represented by Formula (4) with an aromatic compound in a solvent containing an alcohol, 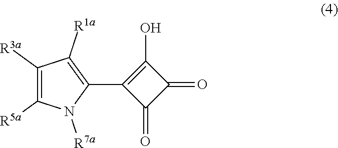
in Formula (4), R^{1a}, R^{3a}, R^{5a}, and R^{7a} each independently represent a hydrogen atom or a substituent, and R^{1a} and R^{3a}, R^{3a} and R^{5a}, and R^{5a} and R^{7a} each may be bonded to each other to form a ring, provided that at least one of R^{1a}, R^{3a}, R^{5a}, or R^{7a} represents a group which includes a polymerizable group having an ethylenically unsaturated bond.

19. A production method of the compound according to claim 2, the production method comprising:
reacting a compound represented by Formula (4) with a compound represented by Formula (5) in a solvent containing an alcohol, 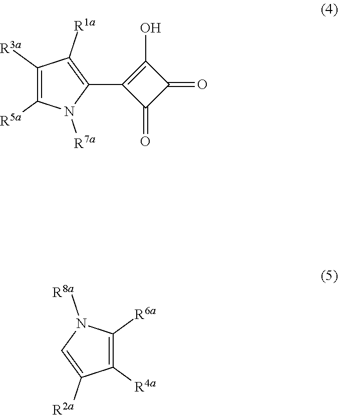
in Formula (4) and Formula (5), R^{1a}, R^{2a}, R^{3a}, R^{4a}, R^{5a}, R^{6a}, R^{7a}, and R^{8a} each independently represent a hydrogen atom or a substituent, and R^{1a} and R^{3a}, R^{2a} and R^{4a}, R^{3a} and R^{5a}, R^{4a} and R^{6a}, R^{5a} and R^{7a}, and R^{6a} and R^{8a} each may be bonded to each other to form a ring, provided that at least one of R^{1a}, R^{2a}, R^{3a}, R^{4a}, R^{5a}, R^{6a}, R^{7a}, or R^{8a} represents a group which includes a polymerizable group having an ethylenically unsaturated bond.

20. A production method of the compound according to claim 2, the production method comprising:
reacting a compound represented by Formula (6) with a compound represented by Formula (7) in a solvent containing an alcohol, 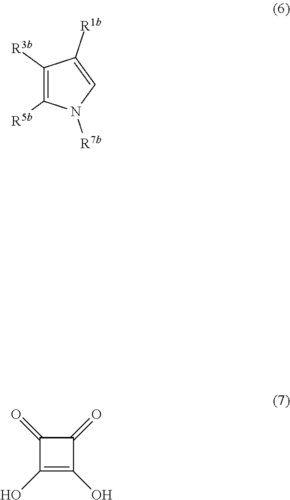
in Formula (6), R^{1b}, R^{3b}, R^{5b}, and R⁷% each independently represent a hydrogen atom or a substituent, and R^{1b} and R^{3b}, R^{3b} and R^{5b}, and R^{5b} and R^{7b} each may be bonded to each other to form a ring, provided that at least one of R^{1b}, R^{3b}, R^{5b}, or R^{7b} represents a group which includes a polymerizable group having an ethylenically unsaturated bond.
